(19) United States
(12) Patent Application Publication
(10) Pub. No.: US 2011/0009368 A1
(43) Pub. Date: Jan. 13, 2011

(54) SOLID FORMS OF TENOFOVIR DISOPROXIL

(75) Inventor: Evanthia Dova, Amsterdam (NL)

Correspondence Address: HOFFMANN & BARON, LLP, 6900 JERICHO TURNPIKE, SYOSSET, NY, 11791, US

(73) Assignee: ULTIMORPHIX TECHNOLOGIES B.V., Amsterdam (NL)

(21) Appl. No.: 12/747,234

(22) PCT Filed: Dec. 11, 2008
(86) PCT No.: PCT/EP2008/010826

§ 371 Date: Sep. 9, 2010

Related U.S. Application Data
(62) Division of application No. 61/013,078, filed on Dec. 12, 2007.

(51) Int. Cl. A61K 31/675; C07F 9/6561; A61P 31/18

(52) U.S. Cl. 514/81; 544/244

(57) ABSTRACT

The Present Invention Provides Tenofovir Disoproxil Succinate, Tenofovir Disoproxil L-Tartrate, Tenofovir Disoproxil oxalate, Tenofovir disoproxil saccharate, Tenofovir disoproxil citrate, Tenofovir disoproxil salicylate and various solid forms thereof, methods for the preparation thereof and their use in pharmaceutical applications, in particular in anti-HIV medicaments. The forms of Tenofovir disoproxil can be used in combination with other anti-HIV medicaments such as Efavirenz and Emtricitabine.

### CROSS-REFERENCE TO RELATED APPLICATIONS

This application is the National Stage of International Application No. PCT/EP2008/010826, filed Dec. 11, 2008, which claims the benefit of U.S. Provisional Application No. 61/013,078, filed Dec. 12, 2007, the contents of which is incorporated by reference herein.

### FIELD OF THE INVENTION

The present invention relates to novel solid forms of Tenofovir disoproxil, in particular combinations of Tenofovir disoproxil with weak organic acids, methods for their preparation and their formulation and application in the field of medicine, in particular antiviral medicines.

### BACKGROUND OF THE INVENTION

Tenofovir disoproxil fumarate (also known as Viread®, Tenofovir DF, Tenofovir disoproxil, TDF, Bis-POC-PMPA, 9-[(R)-2-[[bis[[(isopropoxycarbonyl)oxy]methoxy]phosphinyl]methoxy]propyl]adenine (U.S. Pat. Nos. 5,935,946, 5,922,695, 5,977,089, 6,043,230, 6,069,249) is a prodrug of Tenofovir.

The chemical name of Tenofovir disoproxil fumarate is 9-[(R)-2-[[bis[[(isopropoxycarbonyl)oxy]methoxy]phosphinyl]methoxy]propyl]adenine fumarate (1:1). The CAS Registry number is 202138-50-9. It has a molecular formula of C19H30N5O10P. C4H4O4 and a molecular weight of 635.52. It has the following structural formula:

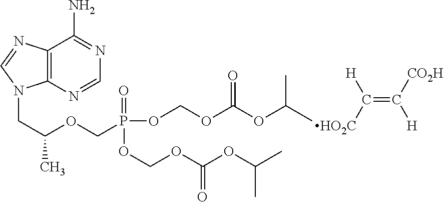

Tenofovir disoproxil fumarate (DF) is a nucleotide reverse transcriptase inhibitor approved in the United States for the treatment of HIV-1 infection in combination with other antiretroviral agents. Tenofovir disoproxil DF is available as Viread® (Gilead Science, Inc.).

Among the anti-HIV drugs which have been developed are those which target the HIV reverse transcriptase (RT) enzyme or protease enzyme, both of which enzymes are necessary for the replication of the virus. Examples of RT inhibitors include nucleoside/nucleotide RT inhibitors (NRTIs) and non-nucleoside RT inhibitors (NNRTIs). Currently, HIV-infected patients are routinely being treated with three-drug combinations. Regimens containing (at least) three NRTIs; two NRTIs in combination with one or two protease inhibitors (PI)(s); or two NRTIs in combination with a NNRTI, are widely used. When two or more PIs are used in these combinations, one of the PIs is often ritonavir, given at a low sub-therapeutic dose, which acts as an effective inhibitor of the elimination of the other PI(s) in the regimen, resulting in maximal suppression of the virus and thereby reducing the emergence of resistance.

Clinical studies have shown that three-drug combinations of these anti-HIV drugs are much more effective than one drug used alone or two-drug combinations in preventing disease progression and death. Numerous studies of drug combinations with various combinations of such drugs have established that such combinations greatly reduce disease progression and deaths in people with HIV infections. The name now commonly given to combinations of anti-HIV drugs is HAART (Highly Active Anti-Retroviral Therapy).

Tenofovir DF is described inter alia in WO99/05150 and EP998480. This crystalline form is characterised as having XRPD peaks at about 4.9, 10.2, 10.5, 18.2, 20.0, 21.9, 24.0, 25.0, 25.5, 27.8, 30.1 and 30.4. Furthermore these crystals are described as opaque or off-white and exhibit a DSC absorption peak at about 118° C. with an onset at about 116° C. and an IR spectrum showing characteristic bands expressed in reciprocal centimetres at approximately 3224, 3107-3052, 2986-2939, 1759, 1678, 1620, 1269 and 1102. Bulk densities have been described of about 0.15-0.30 g/mL, usually about 0.2-0.25 g/mL. Hygroscopicity is well above industry limits of 4%, requiring a desiccant in the packaged product to ensure stability.

After performing a extensive polymorph screen on Tenofovir DF it was found that Tenofovir DF is highly polymorphic and that conversion from one form to other forms might occur under normal processing conditions such as wet granulation.

### SUMMARY OF THE INVENTION

It is a goal of the present inventors to overcome the problems associated with the current fumarate of Tenofovir disoproxil by looking for combinations of Tenofovir disoproxil and other weak organic acids.

The present invention relates to novel solid forms of Tenofovir Disoproxil. The present inventors have identified novel solid forms, herein depicted as succinates, oxalates, saccharates, tartrates, citrates and salicylates of Tenofovir disoproxil. These solid forms may be in the form of salts, polymorphs of salts, co-crystals or polymorphs of co-crystals. The present inventors have found that in particular the succinate ULT-1 has an improved solubility paired with strongly reduced hygroscopicity, compared to the known TDF 1:1.

### DESCRIPTION OF THE DRAWINGS

FIG. 1A illustrates the X-Ray Powder Diffraction pattern of Tenofovir Disoproxil succinate TDSU ULT-1.

FIG. 1B illustrates the DSC thermogram of Tenofovir Disoproxil succinate TDSU ULT-1.

FIG. 1C illustrates the TGA thermogram of Tenofovir Disoproxil succinate TDSU ULT-1.

FIG. 1D illustrates the DVS isotherm plot of Tenofovir Disoproxil succinate TDSU ULT-1.

FIG. 2A illustrates the X-Ray Powder Diffraction pattern of Tenofovir Disoproxil succinate TDSU ULT-2.

FIG. 2B illustrates the DSC thermogram of Tenofovir Disoproxil succinate TDSU ULT-2.

FIG. 2C illustrates the TGA thermogram of Tenofovir Disoproxil succinate TDSU ULT-2.

FIG. 3A illustrates the X-Ray Powder Diffraction pattern of Tenofovir Disoproxil succinate TDSU ULT-3.

FIG. 3C illustrates the TGA thermogram of Tenofovir Disoproxil succinate TDSU ULT-3.

FIG. 4A illustrates the X-Ray Powder Diffraction pattern of Tenofovir Disoproxil tartrate TDTA ULT-1.

FIG. 4B illustrates the DSC thermogram of Tenofovir Disoproxil tartrate TDTA ULT-1.

FIG. 4C illustrates the TGA thermogram of Tenofovir Disoproxil tartrate TDTA ULT-1.

FIG. 5A illustrates the X-Ray Powder Diffraction pattern of Tenofovir Disoproxil tartrate TDTA ULT-2.

FIG. 5C illustrates the TGA thermogram of Tenofovir Disoproxil tartrate TDTA ULT-2.

FIG. 6A illustrates the X-Ray Powder Diffraction pattern of Tenofovir Disoproxil tartrate TDTA ULT-3.

FIG. 6B illustrates the DSC thermogram of Tenofovir Disoproxil tartrate TDTA ULT-3.

FIG. 6C illustrates the TGA thermogram of Tenofovir Disoproxil tartrate TDTA ULT-3.

FIG. 7A illustrates the X-Ray Powder Diffraction pattern of Tenofovir Disoproxil tartrate TDTA ULT-4.

FIG. 8A illustrates the X-Ray Powder Diffraction pattern of Tenofovir Disoproxil oxalate TDOX ULT-1.

FIG. 8B illustrates the DSC thermogram of Tenofovir Disoproxil oxalate TDOX ULT-1.

FIG. 8C illustrates the TGA thermogram of Tenofovir Disoproxil oxalate TDOX ULT-1.

FIG. 9A illustrates the X-Ray Powder Diffraction pattern of Tenofovir Disoproxil oxalate TDOX ULT-2.

FIG. 9B illustrates the DSC thermogram of Tenofovir Disoproxil oxalate TDOX ULT-2.

FIG. 9C illustrates the TGA thermogram of Tenofovir Disoproxil oxalate TDOX ULT-2.

FIG. 10A illustrates the X-Ray Powder Diffraction pattern of Tenofovir Disoproxil oxalate TDOX ULT-3.

FIG. 10B illustrates the DSC thermogram of Tenofovir Disoproxil oxalate TDOX ULT-3.

FIG. 10C illustrates the TGA thermogram of Tenofovir Disoproxil oxalate TDOX ULT-3.

FIG. 11A illustrates the X-Ray Powder Diffraction pattern of Tenofovir Disoproxil oxalate TDOX ULT-4.

FIG. 12A illustrates the X-Ray Powder Diffraction pattern of Tenofovir Disoproxil saccharate TDSA ULT-1.

FIG. 12B illustrates the DSC thermogram of Tenofovir Disoproxil saccharate TDSA ULT-1.

FIG. 13A illustrates the X-Ray Powder Diffraction pattern of Tenofovir Disoproxil saccharate TDSA ULT-2.

FIG. 13B illustrates the DSC thermogram of Tenofovir Disoproxil saccharate TDSA ULT-2.

FIG. 13C illustrates the TGA thermogram of Tenofovir Disoproxil saccharate TDSA ULT-2.

FIG. 14A illustrates the X-Ray Powder Diffraction pattern of Tenofovir Disoproxil saccharate TDSA ULT-3.

FIG. 14B illustrates the DSC thermogram of Tenofovir Disoproxil saccharate TDSA ULT-3.

FIG. 14C illustrates the TGA thermogram of Tenofovir Disoproxil saccharate TDSA ULT-3.

FIG. 15A illustrates the X-Ray Powder Diffraction pattern of Tenofovir Disoproxil citrate TDCI ULT-1 TDCI ULT-1.

FIG. 16A illustrates the X-Ray Powder Diffraction pattern of Tenofovir disoproxil salicylate TDSY ULT-1.

FIG. 17A illustrates the X-Ray Powder Diffraction pattern of Tenofovir Disoproxil succinate TDSU ULT-4.

FIG. 17B illustrates the DSC thermogram of Tenofovir Disoproxil succinate TDSU ULT-4.

FIG. 17C illustrates the TGA thermogram of Tenofovir Disoproxil succinate TDSU ULT-4.

### DETAILED DESCRIPTION OF THE PREFERRED EMBODIMENTS

### Tenofovir Disoproxil Succinates

Tenofovir Disoproxil Succinate TDSU ULT-1

Thus, in one aspect, the present invention provides crystalline Tenofovir disoproxil succinate, herein defined as TDSU ULT-1 characterised by the selection of at least one, preferably at least two, more preferably at least three, even more preferably at least four, particularly preferred at least five and most preferred six X-ray powder diffraction peaks selected from the group consisting of 4.9, 9.5, 10.3, 11.5, 13.3, 14.7, 17.9, 18.2, 19.1, 24.7, 29.8 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group.

In another embodiment TDSU ULT-1 can be characterised by the following set of XRPD peaks (Table 1) and, optionally, by the associated intensities:

TABLE 1

| | Suc2 | | Preferred embodiment | |
|---|---|---|---|---|
| Peak ID | Angle (2θ) | Intensity* | Angle (2θ) | Intensity* |
| 1 | 4.9 | H | 4.90 | H |
| 2 | 9.5 | M | 9.46 | M |
| 3 | 10.3 | M | 10.30 | M |
| 4 | 11.5 | M | 11.54 | M |
| 5 | 13.3 | L | 13.31 | L |
| 6 | 14.7 | L | 14.74 | L |
| 7 | 17.9 | H | 17.90 | H |
| 8 | 18.2 | H | 18.24 | H |
| 9 | 19.1 | M | 19.14 | M |
| 10 | 24.7 | M | 24.74 | M |
| 11 | 29.8 | L | 29.77 | L |

| | | | |
|---|---|---|---|
| normalised intensity values: | L | 0 | 35 |
| | M | 35 | 60 |
| | H | 60 | 100 |

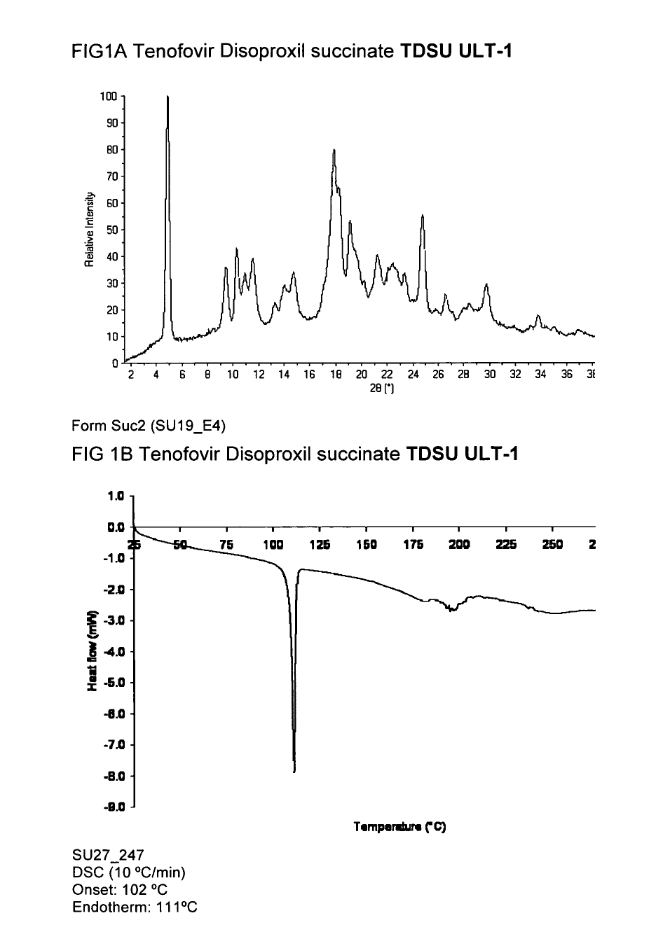

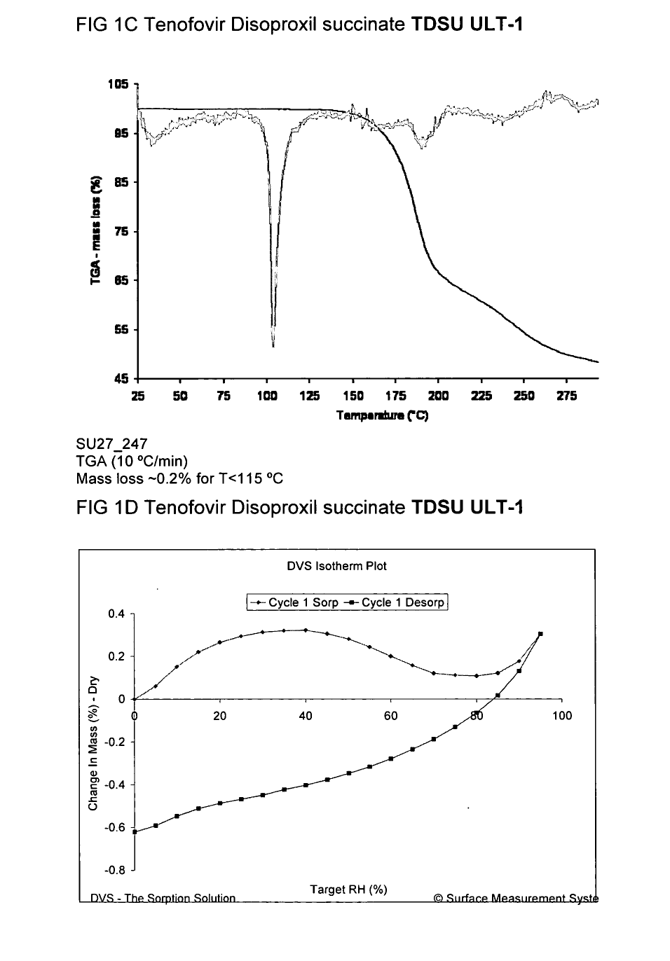

In another embodiment, TDSU ULT-1 can be characterised by an XRPD substantially according to FIG. 1A.

In another embodiment, TDSU ULT-1 can be characterised by an DSC substantially according to FIG. 1B.

In another embodiment, TDSU ULT-1 can be characterised by a TGA substantially according to FIG. 1C.

In another embodiment, TDSU ULT-1 of the present invention can be characterised by DSC with an onset at 102.0° C. and a characterising peak at 111.0° C.

The present invention in one aspect relates to a method for the preparation of the crystalline form of Tenofovir Disoproxil Succinate TDSU ULT-1 comprising the steps of dissolving or mixing Tenofovir disoproxil free base and succinic acid in a suitable solvent or mixture thereof as disclosed herein under ‘Solvents’, preferably methanol, ether, acetone, acetonitrile or mixtures thereof (such as 50/50 v/v methanol-ether) and crystallising Tenofovir Disoproxil Succinate TDSU ULT-1 by evaporation of the solvent.

The present invention in another aspect relates to a method for the preparation of the crystalline Tenofovir Disoproxil Succinate TDSU ULT-1 comprising the steps of dissolving or mixing Tenofovir disoproxil free base and succinic acid in a suitable solvent or mixture thereof as disclosed herein under ‘Solvents’, preferably methanol, ether, acetone, acetonitrile or mixtures thereof (such as 50/50 v/v methanol-ether) and crystallising Tenofovir Disoproxil Succinate TDSU ULT-1 by cooling and/or evaporation crystallization of a saturated solution.

The present invention in one aspect relates to a method for the preparation of the crystalline Tenofovir Disoproxil Succinate TDSU ULT-1 comprising the steps of dissolving or mixing Tenofovir disoproxil free base and succinic acid in a suitable solvent or mixture thereof as disclosed herein under ‘Solvents’, preferably methanol, ether, acetone, acetonitrile or mixtures thereof (such as 50/50 v/v methanol-ether) and crystallising Tenofovir Disoproxil Succinate TDSU ULT-1 by anti-solvent addition as disclosed herein under ‘Solvents’.

The present invention in another aspect relates to a method for the preparation of the crystalline Tenofovir Disoproxil Succinate TDSU ULT-1 comprising the steps of dissolving or mixing Tenofovir disoproxil free base and succinic acid in a suitable solvent or mixture thereof as disclosed herein under ‘Solvents’, preferably methanol, ether, acetone, acetonitrile or mixtures thereof (such as 50/50 v/v methanol-ether) and crystallising Tenofovir Disoproxil Succinate TDSU ULT-1 by slurry crystallisation and/or seed crystallisation.

Tenofovir Disoproxil Succinate TDSU ULT-2

Thus, in one aspect, the present invention provides crystalline Tenofovir disoproxil succinate, herein defined as TDSU ULT-2 characterised by the selection of at least one, preferably at least two, more preferably at least three, even more preferably at least four, particularly preferred at least five and most preferred six X-ray powder diffraction peaks selected from the group consisting of 4.8, 6.6, 9.5, 10.6, 12.6, 13.4, 17.2, 18.4, 19.0, 21.3, 24.1 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group.

In another embodiment TDSU ULT-2 can be characterised by the following set of XRPD peaks (Table 2) and, optionally, by the associated intensities:

TABLE 2

| | Suc3 | | Preferred embodiment | |
|---|---|---|---|---|
| Peak ID | Angle (2θ) | Intensity* | Angle (2θ) | Intensity* |
| 1 | 4.8 | H | 4.78 | H |
| 2 | 6.6 | L | 6.62 | L |
| 3 | 9.5 | L | 9.46 | L |
| 4 | 10.6 | H | 10.58 | H |
| 5 | 12.6 | L | 12.61 | L |
| 6 | 13.4 | L | 13.40 | L |
| 7 | 17.2 | M | 17.20 | M |
| 8 | 18.4 | H | 18.42 | H |
| 9 | 19.0 | M | 19.04 | M |
| 10 | 21.3 | M | 21.32 | M |
| 11 | 24.1 | H | 24.08 | H |

| | | | |
|---|---|---|---|
| normalised intensity values: | L | 0 | 50 |
| | M | 50 | 70 |
| | H | 70 | 100 |

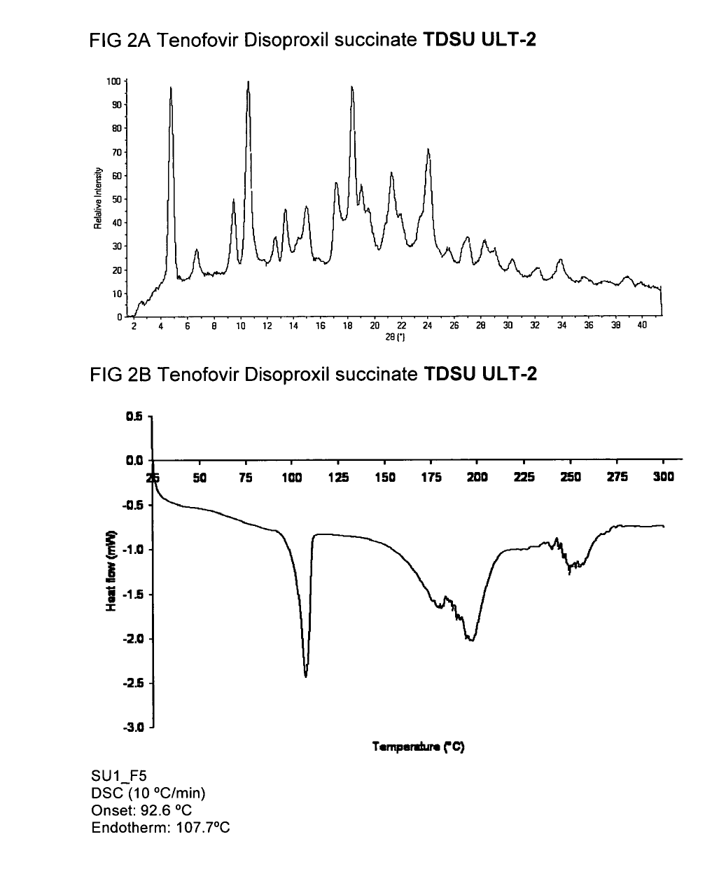

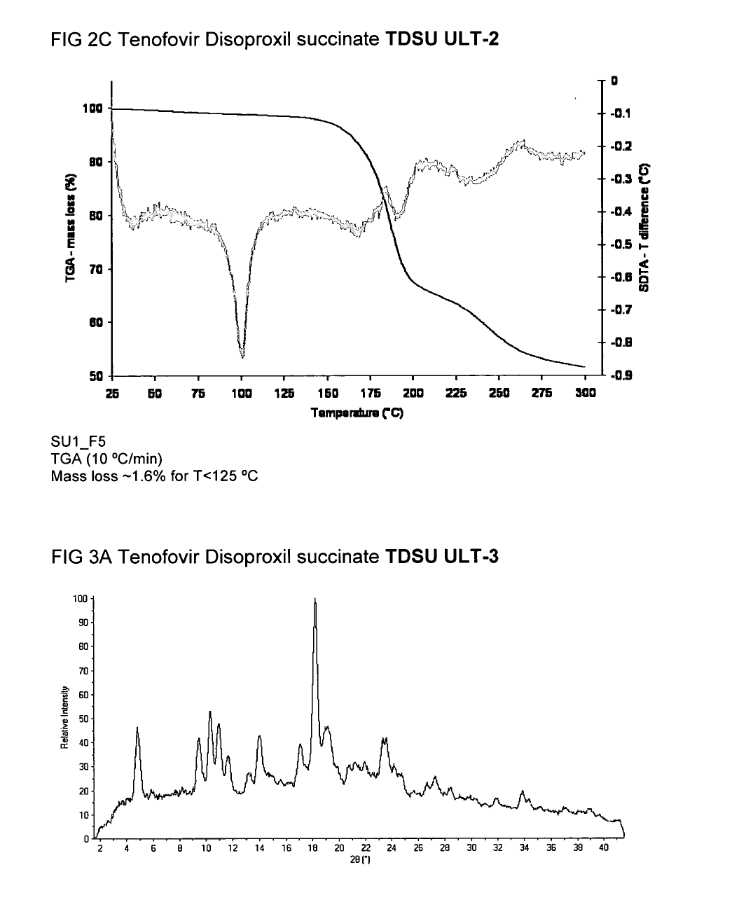

In another embodiment, TDSU ULT-2 can be characterised by an XRPD substantially according to FIG. 2A.

In another embodiment, TDSU ULT-2 can be characterised by an DSC substantially according to FIG. 2B.

In another embodiment, TDSU ULT-2 can be characterised by a TGA substantially according to FIG. 2C.

In another embodiment, TDSU ULT-2 of the present invention can be characterised by DSC with an onset at 92.6° C. and a characterising peak at 107.7° C.

The present invention in one aspect relates to a method for the preparation of the crystalline form of Tenofovir Disoproxil Succinate TDSU ULT-2 comprising the steps of dissolving or mixing Tenofovir disoproxil free base and succinic acid in a suitable solvent or mixture thereof as disclosed herein under ‘Solvents’, preferably chloroform, 1,4-dioxane or mixtures thereof and crystallising Tenofovir Disoproxil Succinate TDSU ULT-2 by evaporation of the solvent.

The present invention in another aspect relates to a method for the preparation of the crystalline Tenofovir Disoproxil Succinate TDSU ULT-2 comprising the steps of dissolving or mixing Tenofovir disoproxil free base and succinic acid in a suitable solvent or mixture thereof as disclosed herein under ‘Solvents’, preferably chloroform, 1,4-dioxane or mixtures thereof and crystallising Tenofovir Disoproxil Succinate TDSU ULT-2 by cooling and/or evaporation crystallization of a saturated solution.

The present invention in one aspect relates to a method for the preparation of the crystalline Tenofovir Disoproxil Succinate TDSU ULT-2 comprising the steps of dissolving or mixing Tenofovir disoproxil free base and succinic acid in a suitable solvent or mixture thereof as disclosed herein under ‘Solvents’, preferably chloroform, 1,4-dioxane or mixtures thereof and crystallising Tenofovir Disoproxil Succinate TDSU ULT-2 by anti-solvent addition as disclosed herein under ‘Solvents’.

The present invention in another aspect relates to a method for the preparation of the crystalline Tenofovir Disoproxil Succinate TDSU ULT-2 comprising the steps of dissolving or mixing Tenofovir disoproxil free base and succinic acid in a suitable solvent or mixture thereof as disclosed herein under ‘Solvents’, preferably chloroform, 1,4-dioxane or mixtures thereof and crystallising Tenofovir Disoproxil Succinate TDSU ULT-2 by slurry crystallisation and/or seed crystallisation.

Tenofovir Disoproxil Succinate TDSU ULT-3

Thus, in one aspect, the present invention provides crystalline Tenofovir disoproxil succinate, herein defined as TDSU ULT-3 characterised by the selection of at least one, preferably at least two, more preferably at least three, even more preferably at least four, particularly preferred at least five and most preferred six X-ray powder diffraction peaks selected from the group consisting of 4.8, 9.5, 10.3, 11.0, 11.7, 13.2, 14.0, 17.1, 18.2, 19.1, 23.3, 23.6 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group.

In another embodiment TDSU ULT-3 can be characterised by the following set of XRPD peaks (Table 3) and, optionally, by the associated intensities:

TABLE 3

| | Suc4 | | Preferred embodiment | |
|---|---|---|---|---|
| Peak ID | Angle (2θ) | Intensity* | Angle (2θ) | Intensity* |
| 1 | 4.8 | M | 4.82 | M |
| 2 | 9.5 | L | 9.46 | L |
| 3 | 10.3 | M | 10.32 | M |
| 4 | 11.0 | M | 10.96 | M |
| 5 | 11.7 | L | 11.66 | L |
| 6 | 13.2 | L | 13.19 | L |
| 7 | 14.0 | L | 14.02 | L |
| 8 | 17.1 | L | 17.06 | L |
| 9 | 18.2 | H | 18.22 | H |
| 10 | 19.1 | M | 19.12 | M |
| 11 | 23.3 | L | 23.32 | L |
| 12 | 23.6 | L | 23.56 | L |

| | | | |
|---|---|---|---|
| normalised intensity values: | L | 0 | 45 |
| | M | 45 | 70 |
| | H | 70 | 100 |

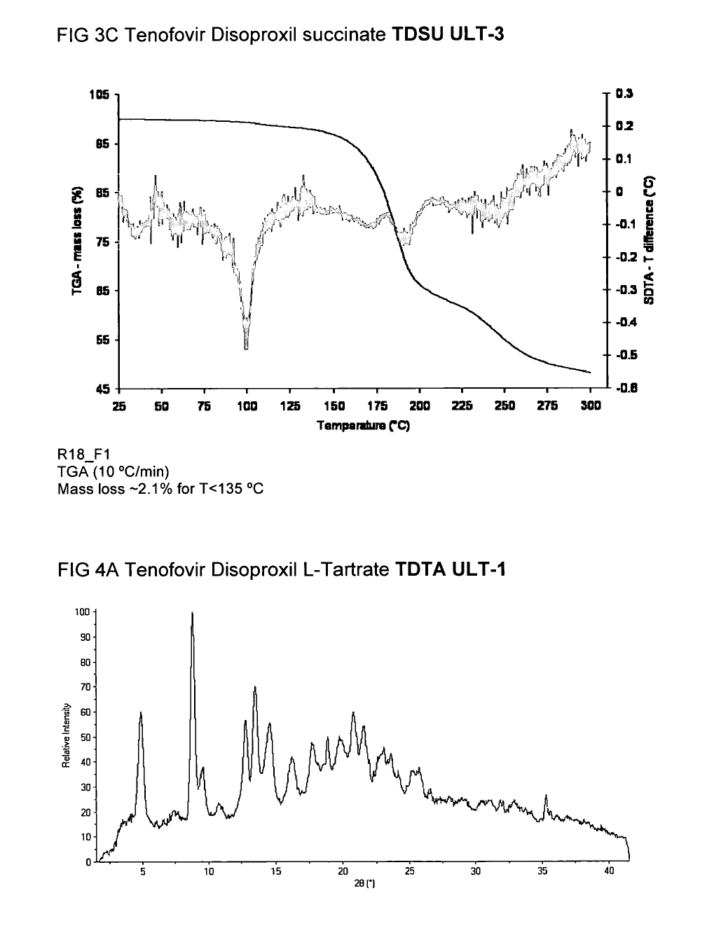

In another embodiment, TDSU ULT-3 can be characterised by an XRPD substantially according to FIG. 3A.

In another embodiment, TDSU ULT-3 can be characterised by a TGA substantially according to FIG. 3C.

From the thermal analysis, it is concluded that solid TDSU ULT-3 is anhydrous.

The present invention in one aspect relates to a method for the preparation of the crystalline form of Tenofovir Disoproxil succinate TDSU ULT-3 comprising the steps of dissolving or mixing Tenofovir disoproxil free base and succinic acid in a suitable solvent or mixture thereof as disclosed herein under ‘Solvents’, preferably acetone and crystallising Tenofovir Disoproxil succinate TDSU ULT-3 by evaporation of the solvent.

The present invention in another aspect relates to a method for the preparation of the crystalline Tenofovir Disoproxil succinate TDSU ULT-3 comprising the steps of dissolving or mixing Tenofovir disoproxil free base and succinic acid in a suitable solvent or mixture thereof as disclosed herein under ‘Solvents’, preferably acetone, and crystallising Tenofovir Disoproxil succinate TDSU ULT-3 by cooling and/or evaporation crystallization of a saturated solution.

The present invention in one aspect relates to a method for the preparation of the crystalline Tenofovir Disoproxil succinate TDSU ULT-3 comprising the steps of dissolving or mixing Tenofovir disoproxil free base and succinic acid in a suitable solvent or mixture thereof as disclosed herein under ‘Solvents’, preferably acetone, and crystallising Tenofovir Disoproxil succinate TDSU ULT-3 by anti-solvent addition as disclosed herein under ‘Solvents’.

The present invention in another aspect relates to a method for the preparation of the crystalline Tenofovir Disoproxil succinate TDSU ULT-3 comprising the steps of dissolving or mixing Tenofovir disoproxil free base and succinic acid in a suitable solvent or mixture thereof as disclosed herein under ‘Solvents’, preferably acetone and crystallising Tenofovir Disoproxil succinate TDSU ULT-3 by slurry crystallisation and/or seed crystallisation.

### Tenofovir Disoproxil Tartrates

Tenofovir Disoproxil Tartrate TDTA ULT-1

Thus, in one aspect, the present invention provides crystalline Tenofovir disoproxil L-tartrate herein defined as TDTA ULT-1, characterised by the selection of at least one, preferably at least two, more preferably at least three, even more preferably at least four, particularly preferred at least five and most preferred six X-ray powder diffraction peaks selected from the group consisting of 4.9, 8.8, 9.6, 12.8, 13.5, 14.6, 16.2, 18.9, 20.8, 21.5, 22.3 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group.

In another embodiment TDTA ULT-1 can be characterised by the following set of XRPD peaks (Table 4) and, optionally, by the associated intensities:

TABLE 4

| | LTar1 | | Preferred embodiment | |
|---|---|---|---|---|
| Peak ID | Angle (2θ) | Intensity* | Angle (2θ) | Intensity* |
| 1 | 4.9 | M | 4.86 | M |
| 2 | 8.8 | H | 8.82 | H |
| 3 | 9.6 | L | 9.56 | L |
| 4 | 12.8 | M | 12.80 | M |
| 5 | 13.5 | M | 13.54 | M |
| 6 | 14.6 | M | 14.60 | M |
| 7 | 16.2 | L | 16.21 | L |
| 8 | 18.9 | L | 18.88 | L |
| 9 | 20.8 | M | 20.78 | M |
| 10 | 21.5 | M | 21.54 | M |
| 11 | 22.3 | L | 22.30 | L |

| | | | |
|---|---|---|---|
| normalised intensity values: | L | 0 | 50 |
| | M | 50 | 70 |
| | H | 70 | 100 |

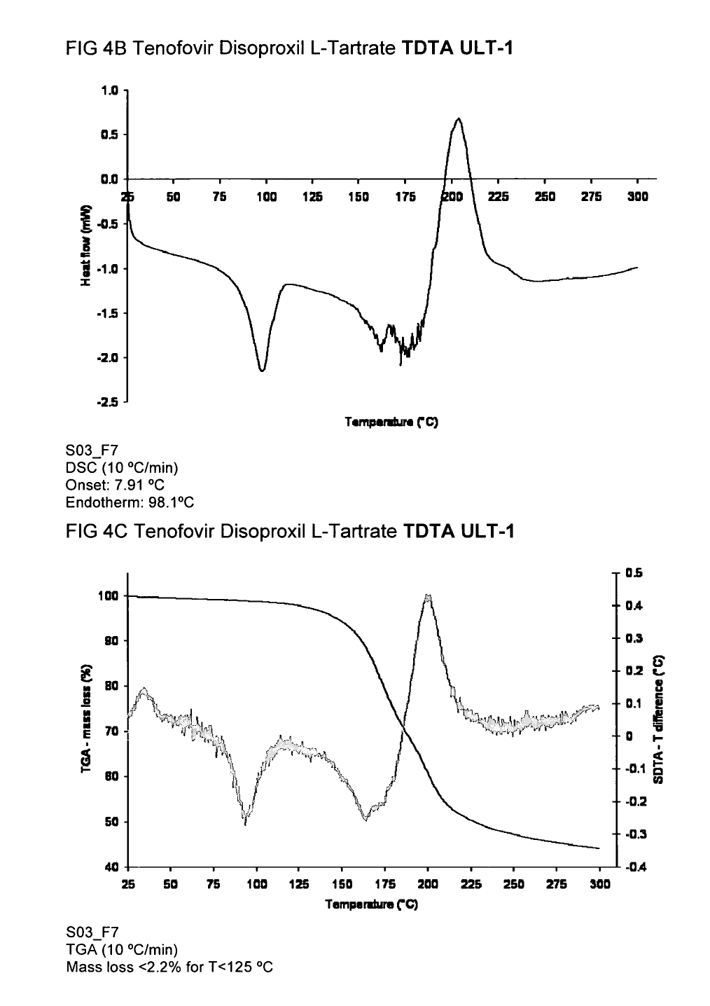

In another embodiment TDTA ULT-1 can be characterised by an XRPD substantially according to FIG. 4A.

In another embodiment, TDTA ULT-1 can be characterised by an DSC substantially according to FIG. 4B.

In another embodiment, TDTA ULT-1 can be characterised by a TGA substantially according to FIG. 4C:

In another embodiment, TDTA ULT-1 of the present invention can be characterised by DSC with an onset at 79.0.° C. and a characterising peak at 98.1° C.

The present invention in one aspect relates to a method for the preparation of the crystalline form of Tenofovir Disoproxil tartrate TDTA ULT-1 comprising the steps of dissolving or mixing Tenofovir disoproxil free base and tartaric acid in a suitable solvent or mixture thereof as disclosed herein under ‘Solvents’, preferably chloroform, acetonitrile or mixtures thereof and crystallising Tenofovir Disoproxil tartrate TDTA ULT-1 by evaporation of the solvent.

The present invention in another aspect relates to a method for the preparation of the crystalline Tenofovir Disoproxil tartrate TDTA ULT-1 comprising the steps of dissolving or mixing Tenofovir disoproxil free base and tartaric acid in a suitable solvent or mixture thereof as disclosed herein under ‘Solvents’, preferably chloroform, acetonitrile or mixtures thereof, and crystallising Tenofovir Disoproxil tartrate TDTA ULT-1 by cooling and/or evaporation crystallization of a saturated solution.

The present invention in one aspect relates to a method for the preparation of the crystalline Tenofovir Disoproxil tartrate TDTA ULT-1 comprising the steps of dissolving or mixing Tenofovir disoproxil free base and tartaric acid in a suitable solvent or mixture thereof as disclosed herein under ‘Solvents’, preferably chloroform, acetonitrile or mixtures thereof and crystallising Tenofovir Disoproxil tartrate TDTA ULT-1 by anti-solvent addition as disclosed herein under ‘Solvents’.

The present invention in another aspect relates to a method for the preparation of the crystalline Tenofovir Disoproxil tartrate TDTA ULT-1 comprising the steps of dissolving or mixing Tenofovir disoproxil free base and tartaric acid in a suitable solvent or mixture thereof as disclosed herein under ‘Solvents’, preferably chloroform, acetonitrile or mixtures thereof and crystallising Tenofovir Disoproxil tartrate TDTA ULT-1 by slurry crystallisation and/or seed crystallisation.

Tenofovir Disoproxil Tartrate TDTA ULT-2

Thus, in one aspect, the present invention provides crystalline Tenofovir disoproxil L-tartrate herein defined as TDTA ULT-2, characterised by characterised by the selection of at least one, preferably at least two, more preferably at least three, even more preferably at least four, particularly preferred at least five and most preferred six X-ray powder diffraction peaks selected from the group consisting of 5.2, 7.8, 8.8, 9.1, 10.4, 11.8, 12.9, 13.7, 14.8, 15.9, 16.4, 18.2, 20.4, 21.2, 22.4, 24.0 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve, more preferably at least thirteen, even more preferably at least fourteen, particularly preferred at least fifteen and most preferred sixteen X-ray powder diffraction peaks are selected from the above group.

In another embodiment TDTA ULT-2 can be characterised by the following set of XRPD peaks (Table 5) and, optionally, by the associated intensities:

TABLE 5

| | LTar4 | | Preferred embodiment | |
|---|---|---|---|---|
| Peak ID | Angle (2θ) | Intensity* | Angle (2θ) | Intensity* |
| 1 | 5.2 | M | 5.20 | M |
| 2 | 7.8 | L | 7.84 | L |
| 3 | 8.8 | M | 8.80 | M |
| 4 | 9.1 | M | 9.14 | M |
| 5 | 10.4 | L | 10.38 | L |
| 6 | 11.8 | L | 11.84 | L |
| 7 | 12.9 | M | 12.86 | M |
| 8 | 13.7 | H | 13.66 | H |
| 9 | 14.8 | L | 14.84 | L |
| 10 | 15.9 | M | 15.88 | M |
| 11 | 16.4 | L | 16.36 | L |
| 12 | 18.2 | M | 18.18 | M |
| 13 | 20.4 | M | 20.38 | M |
| 14 | 21.2 | M | 21.24 | M |
| 15 | 22.4 | M | 22.42 | M |
| 16 | 24.0 | L | 24.04 | L |

| | | | |
|---|---|---|---|
| normalised intensity values: | L | 0 | 50 |
| | M | 50 | 70 |
| | H | 70 | 100 |

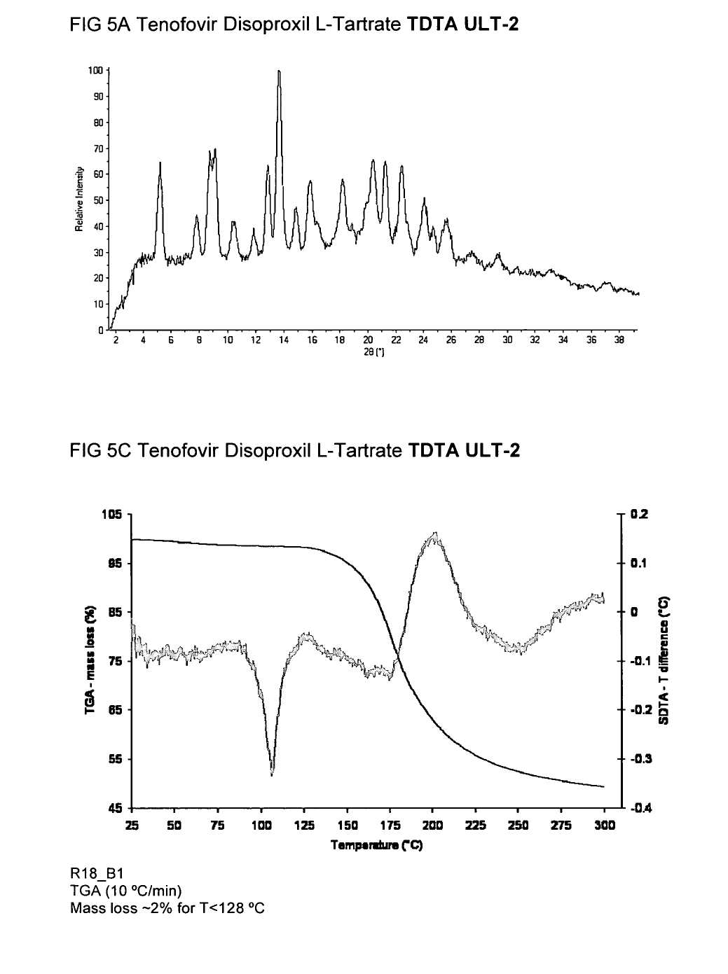

In another embodiment TDTA ULT-2 can be characterised by an XRPD substantially according to FIG. 5A.

In another embodiment, TDTA ULT-2 can be characterised by a TGA substantially according to FIG. 5C.

From the thermal analysis, it is concluded that solid TDTA ULT-2 is anhydrous.

The present invention in one aspect relates to a method for the preparation of the crystalline form of Tenofovir Disoproxil tartrate TDTA ULT-2 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-2 by evaporation of the solvent.

The present invention in another aspect relates to a method for the preparation of the crystalline Tenofovir Disoproxil tartrate TDTA ULT-2 comprising the steps of dissolving or mixing Tenofovir disoproxil free base and tartaric acid in a suitable solvent or mixture thereof as disclosed herein under ‘Solvents’, preferably acetonitrile, and crystallising Tenofovir Disoproxil tartrate TDTA ULT-2 by cooling and/or evaporation crystallization of a saturated solution.

The present invention in one aspect relates to a method for the preparation of the crystalline Tenofovir Disoproxil tartrate TDTA ULT-2 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-2 by anti-solvent addition as disclosed herein under ‘Solvents’.

The present invention in another aspect relates to a method for the preparation of the crystalline Tenofovir Disoproxil tartrate TDTA ULT-2 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-2 by slurry crystallisation and/or seed crystallisation.

### Tenofovir Disoproxil Tartrate TDTA ULT-3

Thus, in one aspect, the present invention provides crystalline Tenofovir disoproxil L-tartrate herein defined as TDTA ULT-3, characterised by characterised by the selection of at least one, preferably at least two, more preferably at least three, even more preferably at least four, particularly preferred at least five and most preferred six X-ray powder diffraction peaks selected from the group consisting of 4.9, 9.0, 11.9, 13.0, 13.8, 15.0, 17.9, 19.3, 20.08, 21, 21.6, 22.5, 23.1, 23.6, 26.5, 28.3 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve, more preferably at least thirteen, even more preferably at least fourteen, particularly preferred at least fifteen and most preferred sixteen X-ray powder diffraction peaks are selected from the above group.

In another embodiment, TDTA ULT-3 can be characterised by the following set of XRPD peaks (Table 6) and, optionally, by the associated intensities:

TABLE 6

| | | | Preferred | |
|---|---|---|---|---|
| | LTar5 | | embodiment | |
| Peak ID | Angle (2θ) | Intensity* | Angle (2θ) | Intensity* |
| 1 | 4.9 | L | 4.86 | L |
| 2 | 9.0 | H | 9.06 | H |
| 3 | 11.9 | L | 11.88 | L |
| 4 | 13.0 | H | 13.04 | H |
| 5 | 13.8 | L | 13.76 | L |
| 6 | 15.0 | M | 14.98 | M |
| 7 | 17.9 | M | 17.86 | M |
| 8 | 19.3 | M | 19.30 | M |
| 9 | 20.08 | M | 20.08 | M |
| 10 | 21 | M | 21.00 | M |
| 11 | 21.6 | L | 21.56 | L |
| 12 | 22.5 | M | 22.46 | M |
| 13 | 23.1 | M | 23.12 | M |
| 14 | 23.6 | L | 23.60 | L |
| 15 | 26.5 | L | 26.54 | L |
| 16 | 28.3 | L | 28.28 | L |

| | | | |
|---|---|---|---|
| normalised intensity values: | L | 0 | 50 |
| | M | 50 | 70 |
| | H | 70 | 100 |

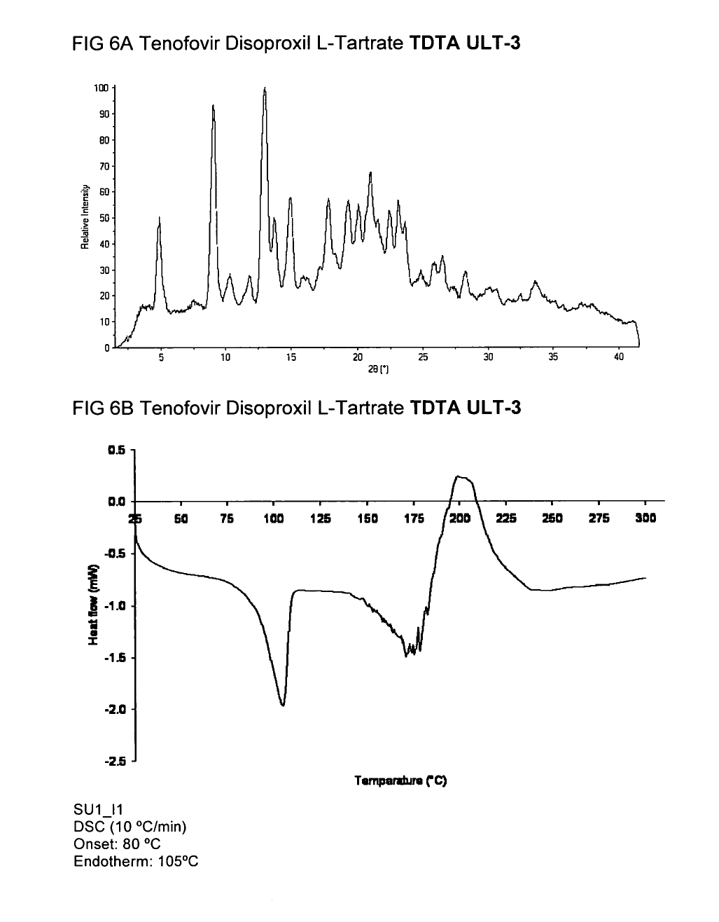

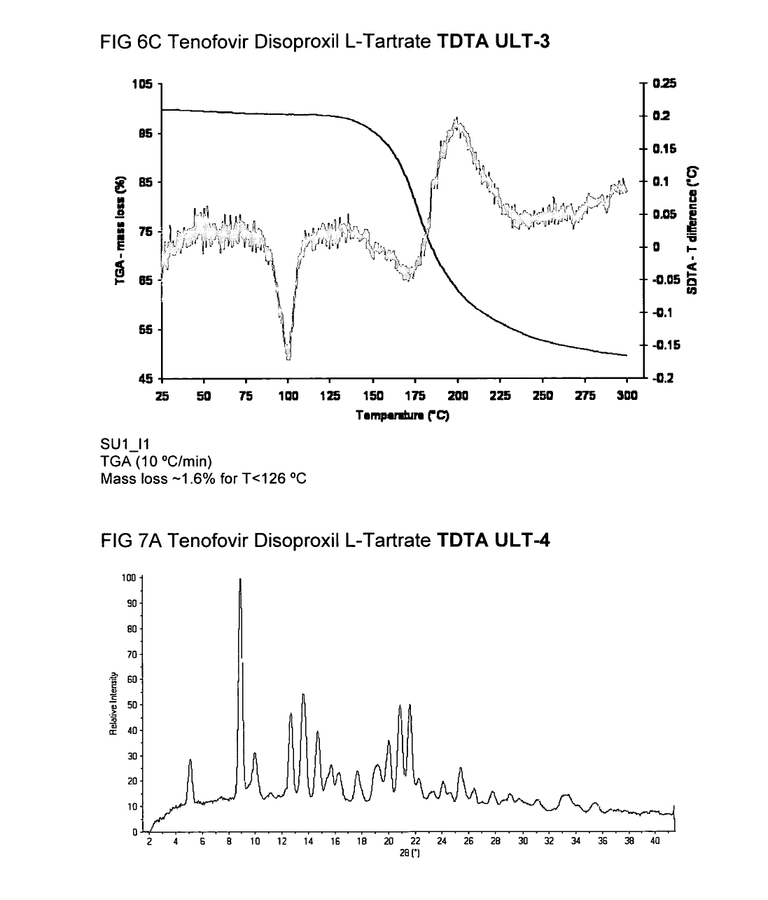

In another embodiment TDTA ULT-3 can be characterised by an XRPD substantially according to FIG. 6A.

In another embodiment, TDTA ULT-3 can be characterised by an DSC substantially according to FIG. 6B.

In another embodiment, TDTA ULT-3 can be characterised by a TGA substantially according to FIG. 6C.

In another embodiment, TDSU ULT-3 of the present invention can be characterised by DSC with an onset at 80° C. and a characterising peak at 105° C.

The present invention in one aspect relates to a method for the preparation of the crystalline form of Tenofovir Disoproxil tartrate TDTA ULT-3 comprising the steps of dissolving or mixing Tenofovir disoproxil free base and tartaric acid in a suitable solvent or mixture thereof as disclosed herein under ‘Solvents’, preferably acetone, tetrahydrofuran or mixtures thereof and crystallising Tenofovir Disoproxil tartrate TDTA ULT-3 by evaporation of the solvent.

The present invention in another aspect relates to a method for the preparation of the crystalline Tenofovir Disoproxil tartrate TDTA ULT-3 comprising the steps of dissolving or mixing Tenofovir disoproxil free base and tartaric acid in a suitable solvent or mixture thereof as disclosed herein under ‘Solvents’, preferably acetone, tetrahydrofuran or mixtures thereof, and crystallising Tenofovir Disoproxil tartrate TDTA ULT-3 by cooling and/or evaporation crystallization of a saturated solution.

The present invention in one aspect relates to a method for the preparation of the crystalline Tenofovir Disoproxil tartrate TDTA ULT-3 comprising the steps of dissolving or mixing Tenofovir disoproxil free base and tartaric acid in a suitable solvent or mixture thereof as disclosed herein under ‘Solvents’, preferably acetone, tetrahydrofuran or mixtures thereof and crystallising Tenofovir Disoproxil tartrate TDTA ULT-3 by anti-solvent addition as disclosed herein under ‘Solvents’.

The present invention in another aspect relates to a method for the preparation of the crystalline Tenofovir Disoproxil tartrate TDTA ULT-3 comprising the steps of dissolving or mixing Tenofovir disoproxil free base and tartaric acid in a suitable solvent or mixture thereof as disclosed herein under ‘Solvents’, preferably acetone, tetrahydrofuran or mixtures thereof and crystallising Tenofovir Disoproxil tartrate TDTA ULT-3 by slurry crystallisation and/or seed crystallisation.

### Tenofovir Disoproxil Tartrate TDTA ULT-4

Thus, in one aspect, the present invention provides crystalline Tenofovir disoproxil L-tartrate herein defined as TDTA ULT-4, characterised by characterised by the selection of at least one, preferably at least two, more preferably at least three, even more preferably at least four, particularly preferred at least five and most preferred six X-ray powder diffraction peaks selected from the group consisting of 5.1, 8.9, 10.0, 12.7, 13.7, 14.7, 15.7, 17.7, 20.0, 20.9, 21.6, 25.4 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In another embodiment, TDTA ULT-4 can be characterised by the following set of XRPD peaks (Table 7) and, optionally, by the associated intensities:

TABLE 7

| | | | Preferred | |
|---|---|---|---|---|
| | LTar6 | | embodiment | |
| Peak ID | Angle (2θ) | Intensity* | Angle (2θ) | Intensity* |
| 1 | 5.1 | L | 5.10 | L |
| 2 | 8.9 | H | 8.92 | H |
| 3 | 10.0 | L | 9.98 | L |
| 4 | 12.7 | M | 12.70 | M |
| 5 | 13.7 | M | 13.66 | M |
| 6 | 14.7 | L | 14.72 | L |
| 7 | 15.7 | L | 15.70 | L |
| 8 | 17.7 | L | 17.68 | L |
| 9 | 20.0 | L | 20.02 | L |
| 10 | 20.9 | M | 20.88 | M |
| 11 | 21.6 | M | 21.62 | M |
| 12 | 25.4 | L | 25.40 | L |

| | | | |
|---|---|---|---|
| normalised intensity values: | L | 0 | 40 |
| | M | 40 | 70 |
| | H | 70 | 100 |

In another embodiment TDTA ULT-4 can be characterised by an XRPD substantially according to FIG. 7A.

The present invention in one aspect relates to a method for the preparation of the crystalline form of Tenofovir Disoproxil tartrate TDTA ULT-4 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-4 by evaporation of the solvent.

The present invention in another aspect relates to a method for the preparation of the crystalline Tenofovir Disoproxil tartrate TDTA ULT-4 comprising the steps of dissolving or mixing Tenofovir disoproxil free base and tartaric acid in a suitable solvent or mixture thereof as disclosed herein under ‘Solvents’, preferably acetonitrile, and crystallising Tenofovir Disoproxil tartrate TDTA ULT-4 by cooling and/or evaporation crystallization of a saturated solution.

The present invention in one aspect relates to a method for the preparation of the crystalline Tenofovir Disoproxil tartrate TDTA ULT-4 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-4 by anti-solvent addition as disclosed herein under ‘Solvents’.

The present invention in another aspect relates to a method for the preparation of the crystalline Tenofovir Disoproxil tartrate TDTA ULT-4 comprising the steps of dissolving or mixing Tenofovir disoproxil free base and tartaric acid in a suitable solvent or mixture thereof as disclosed herein under ‘Solvents’, preferably acetonitrile and crystallising Tenofovir Disoproxil tartrate TDTA ULT-4 by slurry crystallisation and/or seed crystallisation.

### Tenofovir Disoproxil Oxalates

Tenofovir Disoproxil Oxalate TDOX ULT-1

Thus, in one aspect, the present invention provides crystalline Tenofovir disoproxil oxalate herein defined as TDOX ULT-1, characterised by the selection of at least one, preferably at least two, more preferably at least three, even more preferably at least four, particularly preferred at least five and most preferred six X-ray powder diffraction peaks selected from the group consisting of 3.8, 7.6, 9.3, 15.0, 16.4, 17.7, 19.6, 22.6 degrees two-theta+/−0.3 degrees two-theta, preferably +/−0.2 degrees two-theta, more preferably +/−0.1 degrees two-theta, most preferably +/−0.05 degrees two-theta. In a preferred embodiment; at least seven, more preferably at least eight, even more preferably at least nine, particularly preferred at least ten and most preferred eleven X-ray powder diffraction peaks are selected from the above group.

In another embodiment TDOX ULT-1 can be characterised by the following set of XRPD peaks (Table 8) and, optionally, by the associated intensities:

TABLE 8

| | | | Preferred | |
|---|---|---|---|---|
| | Oxa1scr | | embodiment | |
| Peak ID | Angle (2θ) | Intensity* | Angle (2θ) | Intensity* |
| 1 | 3.8 | H | 3.82 | H |
| 2 | 7.6 | H | 7.62 | H |
| 3 | 9.3 | M | 9.26 | M |
| 4 | 15.0 | L | 15.04 | L |
| 5 | 16.4 | H | 16.42 | H |
| 6 | 17.7 | M | 17.74 | M |
| 7 | 19.6 | M | 19.62 | M |
| 8 | 22.6 | L | 22.62 | L |

| | | | |
|---|---|---|---|
| normalised intensity values: | L | 0 | 40 |
| | M | 40 | 70 |
| | H | 70 | 100 |

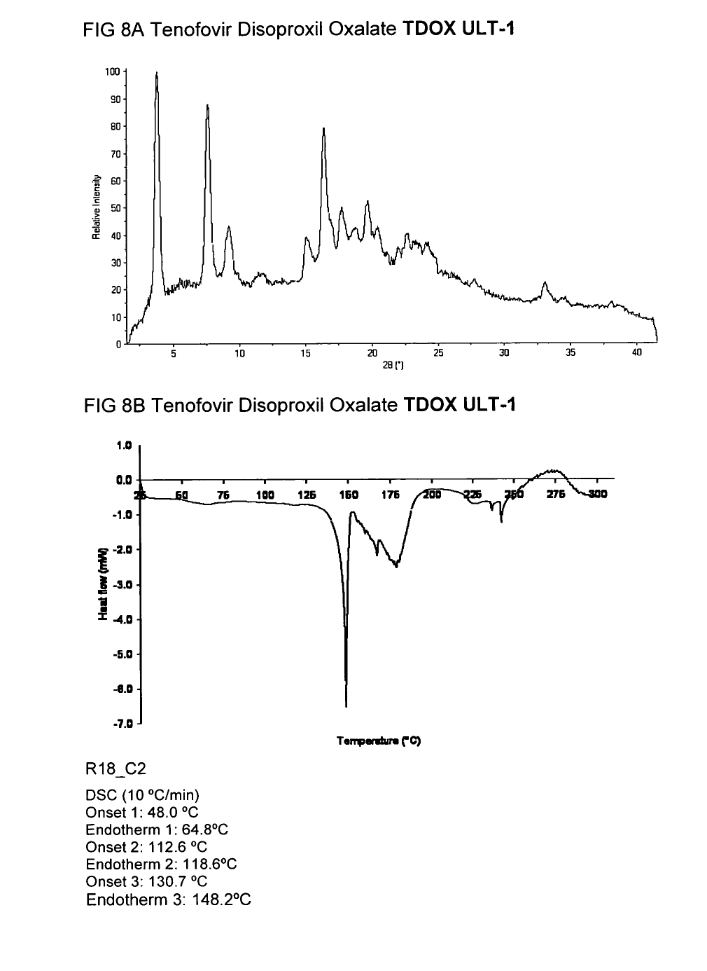

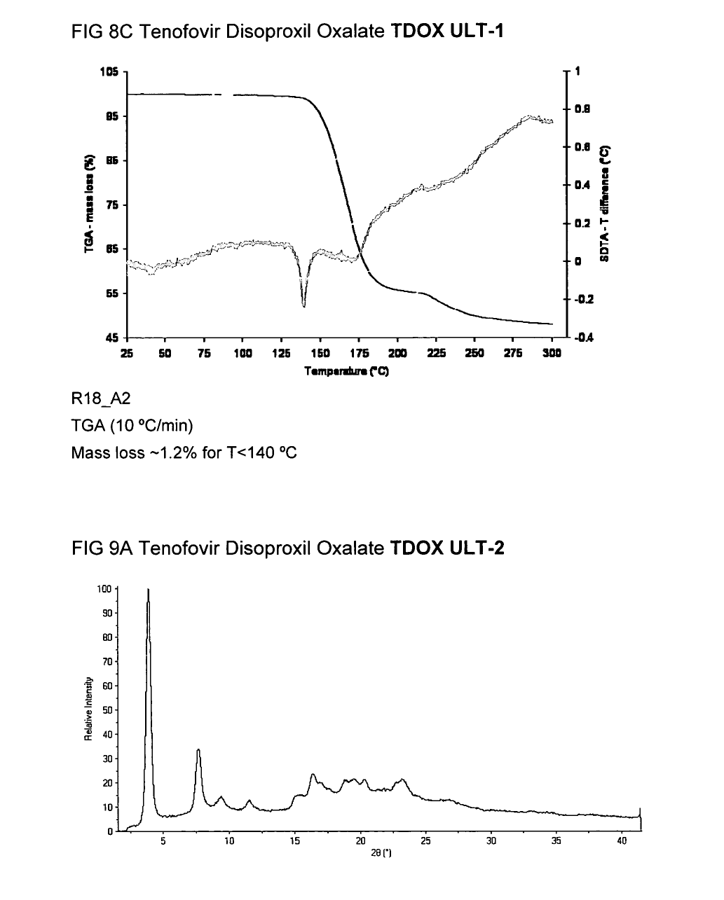

In another embodiment TDOX ULT-1 can be characterised by an XRPD substantially according to FIG. 8A.

In another embodiment, TDOX ULT-1 can be characterised by an DSC substantially according to FIG. 8B.

In another embodiment, TDOX ULT-1 can be characterised by a TGA substantially according to FIG. 8C.

In another embodiment, TDOX ULT-1 of the present invention can be characterised by DSC with an onset at 48.0° C. and a characterising peak at 64.8° C., TDOX ULT-1 of the present invention can be further characterised by DSC with an onset at 112.6 and a characterising peak at 118.6° C. and/or with an onset at 130.7° C. and a characterising peak at 148.2° C.

The present invention in one aspect relates to a method for the preparation of the crystalline form of Tenofovir Disoproxil oxalate TDOX ULT-1 comprising the steps of dissolving or mixing. Tenofovir disoproxil free base and oxalic acid in a suitable solvent or mixture thereof as disclosed herein under ‘Solvents’, preferably chloroform, acetonitrile, methanol, tetrahydrofuran, acetone, water or mixtures thereof and crystallising Tenofovir Disoproxil oxalate TDOX ULT-1 by evaporation of the solvent.

The present invention in another aspect relates to a method for the preparation of the crystalline Tenofovir Disoproxil oxalate TDOX ULT-1 comprising the steps of dissolving or mixing Tenofovir disoproxil free base and oxalic acid in a suitable solvent or mixture thereof as disclosed herein under ‘Solvents’, preferably chloroform, acetonitrile, methanol, tetrahydrofuran, acetone, water or mixtures thereof, and crystallising Tenofovir Disoproxil oxalate TDOX ULT-1 by cooling and/or evaporation crystallization of a saturated solution.

The present invention in one aspect relates to a method for the preparation of the crystalline Tenofovir Disoproxil oxalate TDOX ULT-1 comprising the steps of dissolving or mixing Tenofovir disoproxil free base and oxalic acid in a suitable solvent or mixture thereof as disclosed herein under ‘Solvents’, preferably chloroform, acetonitrile, methanol, tetrahydrofuran, acetone, water or mixtures thereof, and crystallising Tenofovir Disoproxil oxalate TDOX ULT-1 by anti-solvent addition as disclosed herein under ‘Solvents’.

The present invention in another aspect relates to a method for the preparation of the crystalline Tenofovir Disoproxil oxalate TDOX ULT-1 comprising the steps of dissolving or mixing Tenofovir disoproxil free base and oxalic acid in a suitable solvent or mixture thereof as disclosed herein under ‘Solvents’, preferably chloroform, acetonitrile, methanol, tetrahydrofuran, acetone, water or mixtures thereof, and crystallising Tenofovir Disoproxil oxalate TDOX ULT-1 by slurry crystallisation and/or seed crystallisation.

Tenofovir Disoproxil Oxalate TDOX ULT-2

Thus, in one aspect, the present invention provides crystalline Tenofovir disoproxil L-oxalate herein defined as TDOX ULT-2, characterised by characterised by the selection of at least one, preferably at least two, more preferably at least three, even more preferably at least four, particularly preferred at least five and most preferred six X-ray powder diffraction peaks selected from the group consisting of 3.8, 7.6, 9.3, 15.0, 16.4, 17.7, 19.6, 22.6 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight X-ray powder diffraction peaks are selected from the above group.

In another embodiment TDOX ULT-2 can be characterised by the following set of XRPD peaks (Table 9) and, optionally, by the associated intensities:

TABLE 9

| | | | Preferred | |
|---|---|---|---|---|
| | Oxa1 | | embodiment | |
| Peak ID | Angle (2θ) | Intensity* | Angle (2θ) | Intensity* |
| 1 | 3.9 | H | 3.86 | H |
| 2 | 7.7 | L | 7.68 | L |
| 3 | 9.4 | L | 9.40 | L |
| 4 | 11.5 | L | 11.53 | L |
| 5 | 16.4 | L | 16.45 | L |
| 6 | 19.5 | L | 19.50 | L |
| 7 | 20.3 | L | 20.30 | L |
| 8 | 23.1 | L | 23.14 | L |

| | | | |
|---|---|---|---|
| normalised intensity values: | L | 0 | 50 |
| | M | 50 | 70 |
| | H | 70 | 100 |

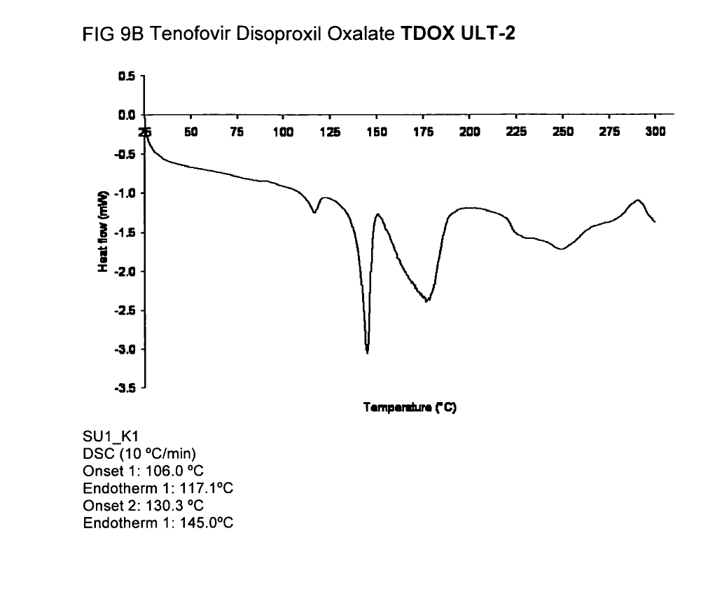

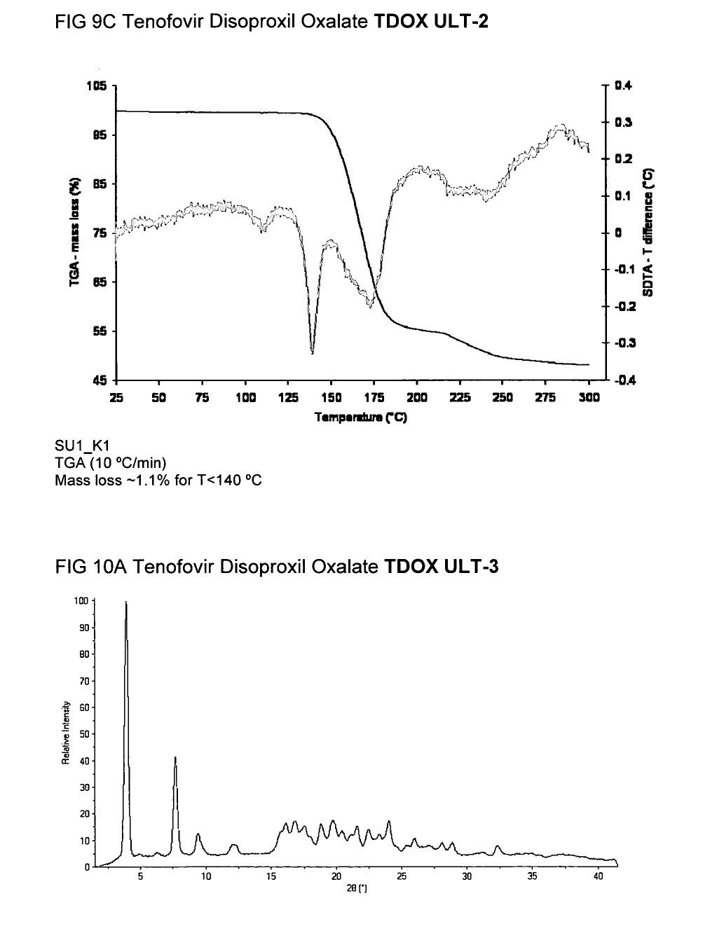

In another embodiment TDOX ULT-2 can be characterised by an XRPD substantially according to FIG. 9A.

In another embodiment, TDOX ULT-2 can be characterised by an DSC substantially according to FIG. 9B.

In another embodiment, TDOX ULT-2 can be characterised by a TGA substantially according to FIG. 9C.

In another embodiment, TDOX ULT-2 of the present invention can be characterised by DSC with an Onset at 106.0° C. and a characterising peak at 117.1° C. TDOX ULT-2 of the present invention can be further characterised by DSC with an onset at 130.3° C. and a characterising peak at 145.0° C. From the thermal analysis, it is concluded that solid TDOX ULT-2 is anhydrous.

The present invention in one aspect relates to a method for the preparation of the crystalline form of Tenofovir Disoproxil oxalate TDOX ULT-2 comprising the steps of dissolving or mixing Tenofovir disoproxil free base and oxalic acid in a suitable solvent or mixture thereof as disclosed herein under ‘Solvents’, preferably acetone, water or mixtures thereof and crystallising Tenofovir Disoproxil oxalate TDOX ULT-2 by evaporation of the solvent.

The present invention in another aspect relates to a method for the preparation of the crystalline Tenofovir Disoproxil oxalate TDOX ULT-2 comprising the steps of dissolving or mixing Tenofovir disoproxil free base and oxalic acid in a suitable solvent or mixture thereof as disclosed herein under ‘Solvents’, preferably acetone, water or mixtures thereof, and crystallising Tenofovir Disoproxil oxalate TDOX ULT-2 by cooling and/or evaporation crystallization of a saturated solution.

The present invention in one aspect relates to a method for the preparation of the crystalline Tenofovir Disoproxil oxalate TDOX ULT-2 comprising the steps of dissolving or mixing Tenofovir disoproxil free base and oxalic acid in a suitable solvent or mixture thereof as disclosed herein under ‘Solvents’, preferably acetone, water or mixtures thereof and crystallising Tenofovir Disoproxil oxalate TDOX ULT-2 by anti-solvent addition as disclosed herein under ‘Solvents’.

The present invention in another aspect relates to a method for the preparation of the crystalline Tenofovir Disoproxil oxalate TDOX ULT-2 comprising the steps of dissolving or mixing Tenofovir disoproxil free base and oxalic acid in a suitable solvent or mixture thereof as disclosed herein under ‘Solvents’, preferably acetone, water or mixtures thereof and crystallising Tenofovir Disoproxil oxalate TDOX ULT-2 by slurry crystallisation and/or seed crystallisation.

### Tenofovir Disoproxil Oxalate TDOX ULT-3

Thus, in one aspect, the present invention provides crystalline Tenofovir disoproxil L-oxalate herein defined as TDOX ULT-3, characterised by characterised by the selection of at least one, preferably at least two, more preferably at least three, even more preferably at least four, particularly preferred at least five and most preferred six X-ray powder diffraction peaks selected from the group consisting of 3.9, 7.7, 9.4, 16.1, 16.8, 17.5, 18.8, 19.7, 21.6, 22.4, 24.0, 28.1 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve X-ray powder diffraction peaks are selected from the above group.

In another embodiment, TDOX ULT-3 can be characterised by the following set of XRPD peaks (Table 10) and, optionally, by the associated intensities:

TABLE 10

| | | | Preferred | |
|---|---|---|---|---|
| | Oxa2 | | embodiment | |
| Peak ID | Angle (2θ) | Intensity* | Angle (2θ) | Intensity* |
| 1 | 3.9 | H | 3.94 | H |
| 2 | 7.7 | M | 7.70 | M |
| 3 | 9.4 | L | 9.37 | L |
| 4 | 16.1 | L | 16.09 | L |
| 5 | 16.8 | L | 16.80 | L |
| 6 | 17.5 | L | 17.54 | L |
| 7 | 18.8 | L | 18.81 | L |
| 8 | 19.7 | L | 19.74 | L |
| 9 | 21.6 | L | 21.56 | L |
| 10 | 22.4 | L | 22.42 | L |
| 11 | 24.0 | L | 24.02 | L |
| 12 | 28.1 | L | 28.11 | L |

| | | | |
|---|---|---|---|
| normalised intensity values: | L | 0 | 40 |
| | M | 40 | 70 |
| | H | 70 | 100 |

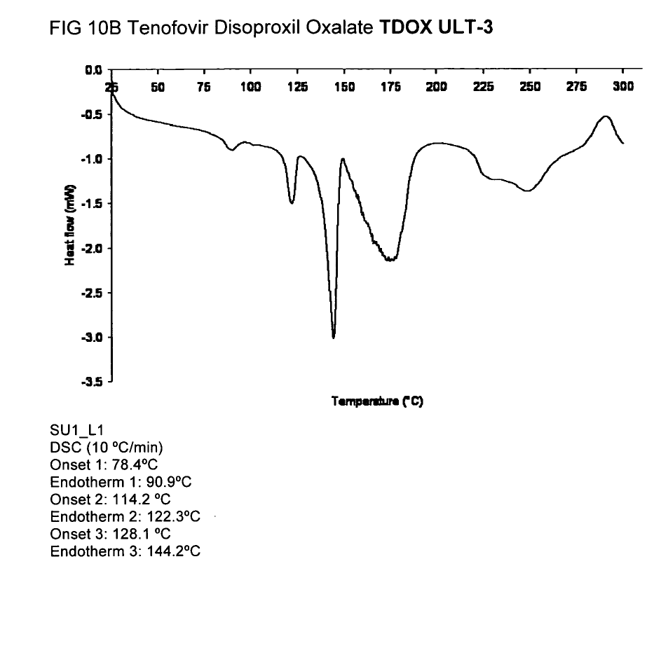

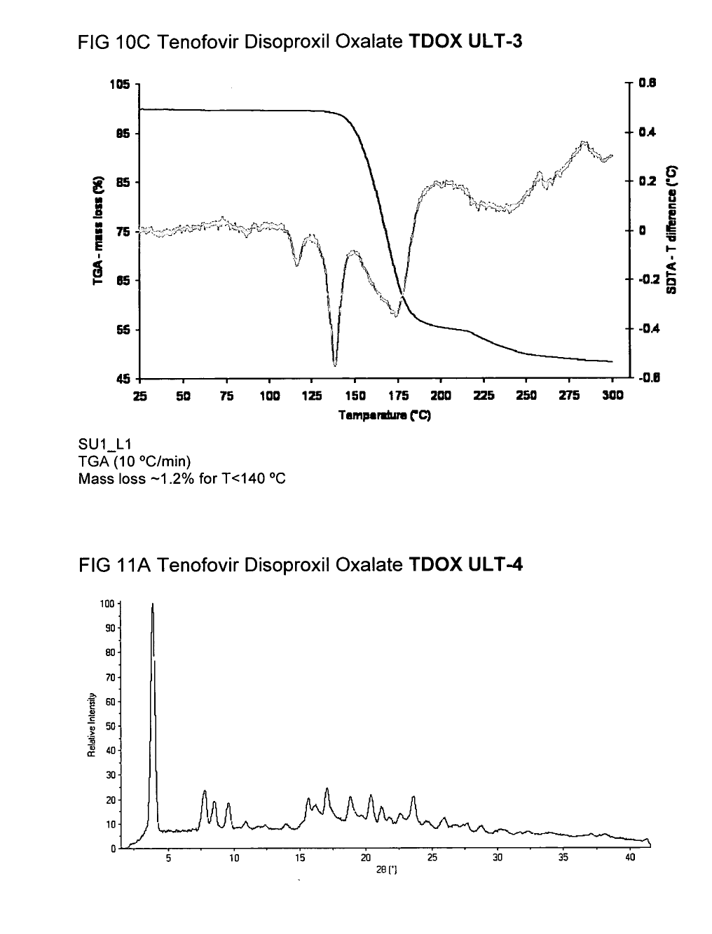

In another embodiment TDOX ULT-3 can be characterised by an XRPD substantially according to FIG. 10A.

In another embodiment, TDOX ULT-3 can be characterised by an DSC substantially according to FIG. 10B.

In another embodiment, TDOX ULT-3 can be characterised by a TGA substantially according to FIG. 10C.

In another embodiment, TDOX ULT-3 of the present invention can be characterised by DSC with an onset at 78.4° C. and a characterising peak at 90.9° C. TDOX ULT-3 of the present invention can also be characterised by DSC with an onset at 114.2° C. and a characterising peak at 122.3° C. TDOX ULT-3 of the present invention can also be characterised by DSC with an onset at 128.1° C. and a characterising peak at 144.2° C. From the thermal analysis, it is concluded that solid TDOX ULT-3 is anhydrous.

The present invention in one aspect relates to a method for the preparation of the crystalline form of Tenofovir Disoproxil oxalate TDOX ULT-3 comprising the steps of dissolving or mixing Tenofovir disoproxil free base and oxalic acid in a suitable solvent or mixture thereof as disclosed herein under ‘Solvents’, preferably water, acetone, 1,4-dioxane or mixtures thereof and crystallising Tenofovir Disoproxil oxalate TDOX ULT-3 by evaporation of the solvent.

The present invention in another aspect relates to a method for the preparation of the crystalline Tenofovir Disoproxil oxalate TDOX ULT-3 comprising the steps of dissolving or mixing Tenofovir disoproxil free base and oxalic acid in a suitable solvent or mixture thereof as disclosed herein under ‘Solvents’, preferably water, acetone, 1,4-dioxane or mixtures thereof, and crystallising Tenofovir Disoproxil oxalate TDOX ULT-3 by cooling and/or evaporation crystallization of a saturated solution.

The present invention in one aspect relates to a method for the preparation of the crystalline Tenofovir Disoproxil oxalate TDOX ULT-3 comprising the steps of dissolving or mixing Tenofovir disoproxil free base and oxalic acid in a suitable solvent or mixture thereof as disclosed herein under ‘Solvents’, preferably water, acetone; 1,4-dioxane or mixtures thereof and crystallising Tenofovir Disoproxil oxalate TDOX ULT-3 by anti-solvent addition as disclosed herein under ‘Solvents’.

The present invention in another aspect relates to a method for the preparation of the crystalline Tenofovir Disoproxil oxalate TDOX ULT-3 comprising the steps of dissolving or mixing Tenofovir disoproxil free base and oxalic acid in a suitable solvent or mixture thereof as disclosed herein under ‘Solvents’, preferably water, acetone, 1,4-dioxane or mixtures thereof and crystallising Tenofovir Disoproxil oxalate TDOX ULT-3 by slurry crystallisation and/or seed crystallisation.

### Tenofovir Disoproxil Oxalate TDOX ULT-4

Thus, in one aspect, the present invention provides crystalline Tenofovir disoproxil L-oxalate herein defined as TDOX ULT-4, characterised by characterised by the selection of at least one, preferably at least two, more preferably at least three, even more preferably at least four, particularly preferred at least five and most preferred six X-ray powder diffraction peaks selected from the group consisting of 3.9, 7.8, 8.5, 9.6, 10.9, 15.7, 17.1, 18.8, 20.4, 23.6 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In another embodiment, TDOX ULT-4 can be characterised by the following set of XRPD peaks (Table 11) and, optionally, by the associated intensities:

TABLE 11

| | | | Preferred | |
|---|---|---|---|---|
| | Oxa3 | | embodiment | |
| Peak ID | Angle (2θ) | Intensity* | Angle (2θ) | Intensity* |
| 1 | 3.9 | H | 3.90 | H |
| 2 | 7.8 | L | 7.81 | L |
| 3 | 8.5 | L | 8.50 | L |
| 4 | 9.6 | L | 9.56 | L |
| 5 | 10.9 | L | 10.9 | L |
| 6 | 15.7 | L | 15.68 | L |
| 7 | 17.1 | L | 17.06 | L |
| 8 | 18.8 | L | 18.83 | L |
| 9 | 20.4 | L | 20.40 | L |
| 10 | 23.6 | L | 23.60 | L |

| | | | |
|---|---|---|---|
| normalised intensity values: | L | 0 | 40 |
| | M | 40 | 70 |
| | H | 70 | 100 |

In another embodiment TDOX ULT-4 can be characterised by an XRPD substantially according to FIG. 11A.

The present invention in one aspect relates to a method for the preparation of the crystalline form of Tenofovir Disoproxil oxalate TDOX ULT-4 comprising the steps of dissolving or mixing Tenofovir disoproxil free base and oxalic acid in a suitable solvent or mixture thereof as disclosed herein under ‘Solvents’, preferably methanol, water or mixtures thereof and crytallising Tenofovir Disoproxil oxalate TDOX ULT-4 by evaporation of the solvent.

The present invention in another aspect relates to a method for the preparation of the crystalline Tenofovir Disoproxil oxalate TDOX ULT-4 comprising the steps of dissolving or mixing Tenofovir disoproxil free base and oxalic acid in a suitable solvent or mixture thereof as disclosed herein under ‘Solvents’, preferably methanol, water or mixtures thereof, and crystallising Tenofovir Disoproxil oxalate TDOX ULT-4 by cooling and/or evaporation crystallization of a saturated solution.

The present invention in one aspect relates to a method for the preparation of the crystalline Tenofovir Disoproxil oxalate TDOX ULT-4 comprising the steps of dissolving or mixing Tenofovir disoproxil free base and oxalic acid in a suitable solvent or mixture thereof as disclosed herein under ‘Solvents’, preferably methanol, water or mixtures thereof and crystallising Tenofovir Disoproxil oxalate TDOX ULT-4 by anti-solvent addition as disclosed herein under ‘Solvents’.

The present invention in another aspect relates to a method for the preparation of the crystalline Tenofovir Disoproxil oxalate TDOX ULT-4 comprising the steps of dissolving or mixing Tenofovir disoproxil free base and oxalic acid in a suitable solvent or mixture thereof as disclosed herein under ‘Solvents’, preferably methanol, water or mixtures thereof and crystallising Tenofovir Disoproxil oxalate TDOX ULT-4 by slurry crystallisation and/or seed crystallisation.

### Tenofovir Disoproxil Saccharate

Tenofovir Disoproxil Saccharate TDSA ULT-1

Thus, in one aspect, the present invention provides crystalline Tenofovir disoproxil L-saccharate herein defined as TDSA ULT-1, characterised by the selection of at least one, preferably at least two, more preferably at least three, even more preferably at least four, particularly preferred at least five and most preferred six X-ray powder diffraction peaks selected from the group consisting of 3.3, 4.1, 7.6, 10.4, 13, 13.6, 17.9, 18.7, 22.7 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine X-ray powder diffraction peaks are selected from the above group.

In another embodiment TDSA ULT-1 can be characterised by the following set of XRPD peaks (Table 12) and, optionally, by the associated intensities:

TABLE 12

| | | | Preferred | |
|---|---|---|---|---|
| | Sac1 | | embodiment | |
| Peak ID | Angle (2θ) | Intensity* | Angle (2θ) | Intensity* |
| 1 | 3.3 | L | 3.34 | L |
| 2 | 4.1 | H | 4.06 | H |
| 3 | 7.6 | L | 7.62 | L |
| 4 | 10.4 | L | 10.40 | L |
| 5 | 13 | L | 13.00 | L |
| 6 | 13.6 | L | 13.57 | L |
| 7 | 17.9 | L | 17.87 | L |
| 8 | 18.7 | L | 18.72 | L |
| 9 | 22.7 | L | 22.72 | L |

| | | | |
|---|---|---|---|
| normalised intensity values: | L | 0 | 40 |
| | M | 40 | 70 |
| | H | 70 | 100 |

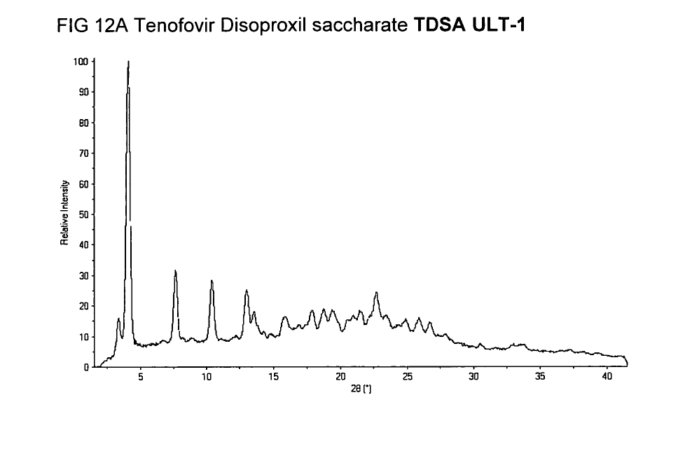

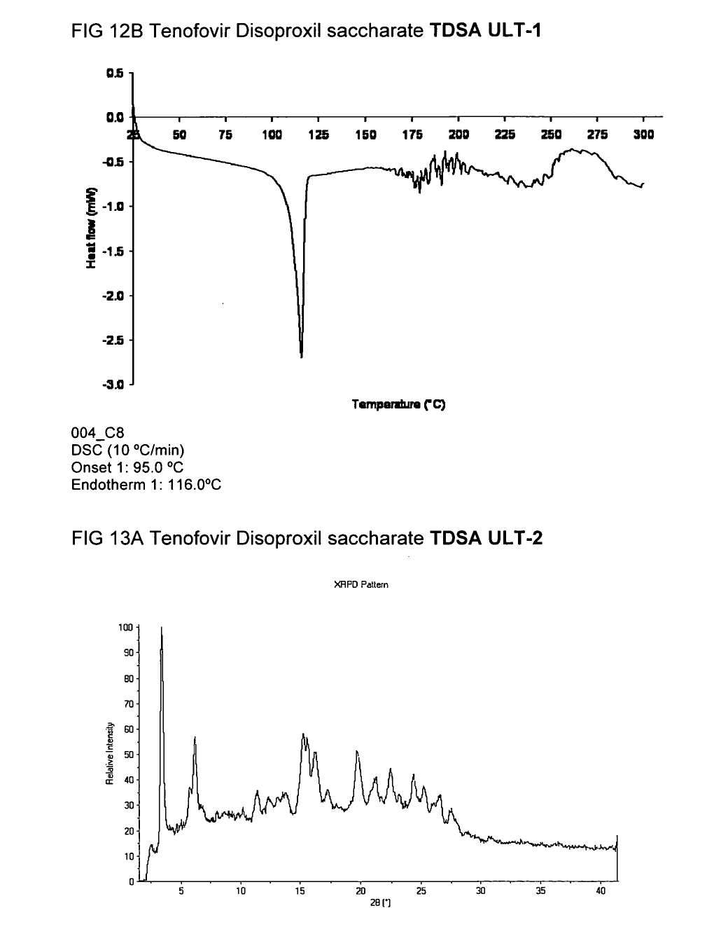

In another embodiment TDSA ULT-1 can be characterised by an XRPD substantially according to FIG. 12A.

In another embodiment, TDSA ULT-1 can be characterised by an DSC substantially according to FIG. 12B.

In another embodiment, TDSA ULT-1 of the present invention can be characterised by DSC with an onset at 95.0° C. and a characterising peak at 116.0° C.

The present invention in one aspect relates to a method for the preparation of the crystalline form of Tenofovir Disoproxil saccharate TDSA ULT-1 comprising the steps of dissolving or mixing Tenofovir disoproxil free base and saccharin in a suitable solvent or mixture thereof as disclosed herein under ‘Solvents’, preferably chloroform, nitromethane, nitroethane or mixtures thereof and crystallising Tenofovir Disoproxil saccharate TDSA ULT-1 by evaporation of the solvent.

The present invention in another aspect relates to a method for the preparation of the crystalline Tenofovir Disoproxil saccharate TDSA ULT-1 comprising the steps of dissolving or mixing Tenofovir disoproxil free base and saccharin in a suitable solvent or mixture thereof as disclosed herein under ‘Solvents’, preferably chloroform, nitromethane, nitroethane or mixtures thereof and crystallising Tenofovir Disoproxil saccharate TDSA ULT-1 by cooling and/or evaporation crystallization of a saturated solution.

The present invention in one aspect relates to a method for the preparation of the crystalline Tenofovir Disoproxil saccharate TDSA ULT-1 comprising the steps of dissolving or mixing Tenofovir disoproxil free base and saccharin in a suitable solvent or mixture thereof as disclosed herein under ‘Solvents’, preferably chloroform, nitromethane, nitroethane or mixtures thereof and crystallising Tenofovir Disoproxil saccharate TDSA ULT-1 by anti-solvent addition as disclosed herein under ‘Solvents’.

The present invention in another aspect relates to a method for the preparation of the crystalline Tenofovir Disoproxil saccharate TDSA ULT-1 comprising the steps of dissolving or mixing Tenofovir disoproxil free base and saccharin in a suitable solvent or mixture thereof as disclosed herein under ‘Solvents’, preferably chloroform, nitromethane, nitroethane or mixtures thereof and crystallising Tenofovir Disoproxil saccharate TDSA ULT-1 by slurry crystallisation and/or seed crystallisation.

Tenofovir Disoproxil Saccharate TDSA ULT-2

Thus, in one aspect, the present invention provides crystalline Tenofovir disoproxil L-saccharate herein defined as TDSA ULT-2, characterised by characterised by the selection of at least one, preferably at least two, more preferably at least three, even more preferably at least four, particularly preferred at least five and most preferred six X-ray powder diffraction peaks selected from the group consisting of 3.4, 6.2, 15.3, 15.6, 16.2, 19.7, 22.4, 24.4 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight X-ray powder diffraction peaks are selected from the above group.

In another embodiment TDSA ULT-2 can be characterised by the following set of XRPD peaks (Table 13) and, optionally, by the associated intensities:

TABLE 13

| | | | Preferred | |
|---|---|---|---|---|
| | Sac2 | | embodiment | |
| Peak ID | Angle (2θ) | Intensity* | Angle (2θ) | Intensity* |
| 1 | 3.4 | H | 3.42 | H |
| 2 | 6.2 | M | 6.18 | M |
| 3 | 15.3 | M | 15.28 | M |
| 4 | 15.6 | M | 15.61 | M |
| 5 | 16.2 | M | 16.22 | M |
| 6 | 19.7 | M | 19.70 | M |
| 7 | 22.4 | L | 22.42 | L |
| 8 | 24.4 | L | 24.36 | L |

| | | | |
|---|---|---|---|
| normalised intensity values: | L | 0 | 50 |
| | M | 50 | 70 |
| | H | 70 | 100 |

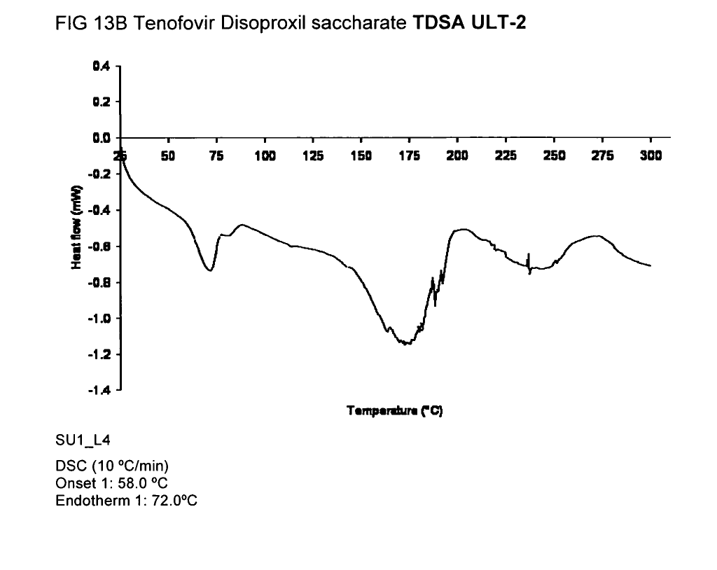

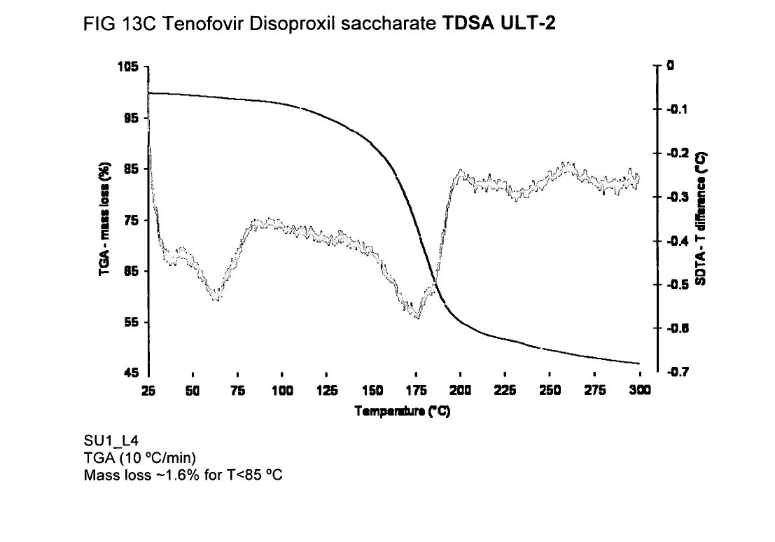

In another embodiment TDSA ULT-2 can be characterised by an XRPD substantially according to FIG. 13A.

The present invention in one aspect relates to a method for the preparation of the crystalline form of Tenofovir Disoproxil saccharate TDSA ULT-2 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-2 by evaporation of the solvent.

The present invention in another aspect relates to a method for the preparation of the crystalline Tenofovir Disoproxil saccharate TDSA ULT-2 comprising the steps of dissolving or mixing Tenofovir disoproxil free base and saccharin in a suitable solvent or mixture thereof as disclosed herein under ‘Solvents’ preferably chloroform, and crystallising Tenofovir Disoproxil saccharate TDSA ULT-2 by cooling and/or evaporation crystallization of a saturated solution.

The present invention in one aspect relates to a method for the preparation of the crystalline Tenofovir Disoproxil saccharate TDSA ULT-2 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-2 by anti-solvent addition as disclosed herein under ‘Solvents’.

The present invention in another aspect relates to a method for the preparation of the crystalline Tenofovir Disoproxil saccharate TDSA ULT-2 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-2 by slurry crystallisation and/or seed crystallisation.

### Tenofovir Disoproxil Saccharate TDSA ULT-3

Thus, in one aspect, the present invention provides crystalline Tenofovir disoproxil L-saccharate herein defined as TDSA ULT-3, characterised by characterised by the selection of at least one, preferably at least two, more preferably at least three, even more preferably at least four, particularly preferred at least five and most preferred six X-ray powder diffraction peaks selected from the group consisting of 3.94, 7.57, 10.42, 12.58, 15.34, 16.46, 17.68, 20.46, 21.94, 24.66 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve X-ray powder diffraction peaks are selected from the above group.

In another embodiment, TDSA ULT-3 can be characterised by the following set of XRPD peaks (Table 14) and, optionally, by the associated intensities:

TABLE 14

| | | | Preferred | |
|---|---|---|---|---|
| | Sac3 | | embodiment | |
| Peak ID | Angle (2θ) | Intensity* | Angle (2θ) | Intensity* |
| 1 | 3.94 | H | 3.94 | H |
| 2 | 7.57 | L | 7.57 | L |
| 3 | 10.42 | L | 10.42 | L |
| 4 | 12.58 | M | 12.58 | M |
| 5 | 15.34 | L | 15.34 | L |
| 6 | 16.46 | L | 16.46 | L |
| 7 | 17.68 | L | 17.68 | L |
| 8 | 20.46 | L | 20.46 | L |
| 9 | 21.94 | M | 21.94 | M |
| 10 | 24.66 | M | 24.66 | M |

| | | | |
|---|---|---|---|
| normalised intensity values: | L | 0 | 40 |
| | M | 40 | 70 |
| | H | 70 | 100 |

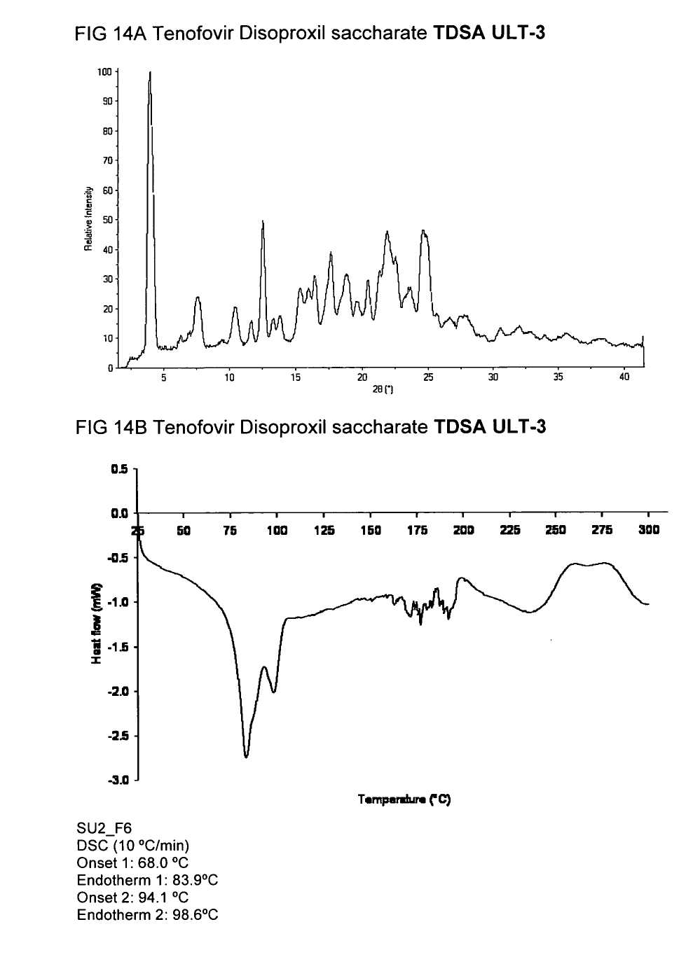

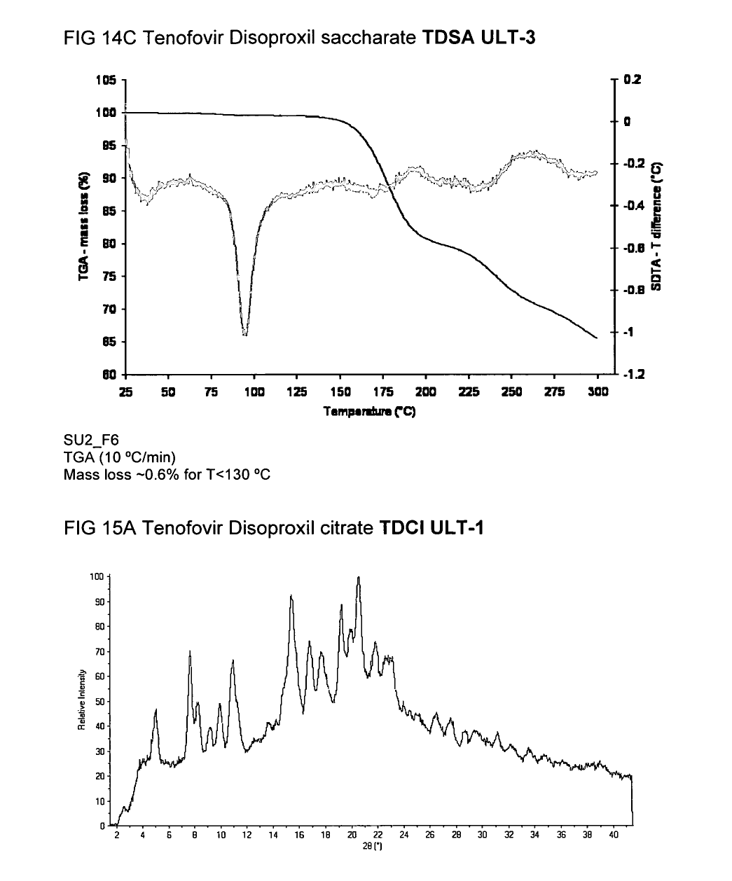

In another embodiment TDSA ULT-3 can be characterised by an XRPD substantially according to FIG. 14A.

In another embodiment, TDSA ULT-3 can be characterised by an DSC substantially according to FIG. 14B.

In another embodiment, TDSA ULT-3 can be characterised by a TGA substantially according to FIG. 14C.

In another embodiment, TDSA ULT-3 of the present invention can be characterised by DSC with an onset at 68.0° C. and a characterising peak at 83.9° C. TDSA ULT-3 of the present invention can also be characterised by DSC with an onset at 94.1° C. and a characterising peak at 98.6° C. From the thermal analysis, it is concluded that solid TDSA ULT-3 is anhydrous.

The present invention in one aspect relates to a method for the preparation of the crystalline form of Tenofovir Disoproxil saccharate TDSA ULT-3 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-3 by evaporation of the solvent.

The present invention in another aspect relates to a method for the preparation of the crystalline Tenofovir Disoproxil saccharate TDSA ULT-3 comprising the steps of dissolving or mixing Tenofovir disoproxil free base and saccharin in a suitable solvent or mixture thereof as disclosed herein under ‘Solvents’, preferably chloroform, and crystallising Tenofovir Disoproxil saccharate TDSA ULT-3 by cooling and/or evaporation crystallization of a saturated solution.

The present invention in one aspect relates to a method for the preparation of the crystalline Tenofovir Disoproxil saccharate TDSA ULT-3 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-3 by anti-solvent addition as disclosed herein under ‘Solvents’.

The present invention in another aspect relates to a method for the preparation of the crystalline Tenofovir Disoproxil saccharate TDSA ULT-3 comprising the steps of dissolving or mixing Tenofovir disoproxil free base and saccharin in a suitable solvent or mixture thereof as disclosed herein under ‘Solvents’, preferably chloroform and crystallising Tenofovir Disoproxil saccharate TDSA ULT-3 by slurry crystallisation and/or seed crystallisation.

### Tenofovir Disoproxil Citrate

Tenofovir Disoproxil Citrate TDCI ULT-1

Thus, in one aspect, the present invention provides crystalline Tenofovir disoproxil citrate TDCI ULT-1 herein defined as TDCI ULT-1, characterised by the selection of at least one, preferably at least two, more preferably at least three, even more preferably at least four, particularly preferred at least five and most preferred six X-ray powder diffraction peaks selected from the group consisting of 5.0, 7.7, 8.2, 10.0, 11.0, 15.4, 16.8, 17.7, 19.2, 20.5, 21.8, 26.5, 27.6 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine X-ray powder diffraction peaks are selected from the above group.

In another embodiment TDCI ULT-1 can be characterised by the following set of XRPD peaks (Table 15) and, optionally, by the associated intensities:

TABLE 15

| | | | Preferred | |
|---|---|---|---|---|
| | Cit2 | | embodiment | |
| Peak ID | Angle (2θ) | Intensity* | Angle (2θ) | Intensity* |
| 1 | 5.0 | L | 5.00 | L |
| 2 | 7.7 | H | 7.66 | H |
| 3 | 8.2 | L | 8.24 | L |
| 4 | 10.0 | L | 9.94 | L |
| 5 | 11.0 | M | 10.96 | M |
| 6 | 15.4 | H | 15.42 | H |
| 7 | 16.8 | H | 16.80 | H |
| 8 | 17.7 | M | 17.66 | M |
| 9 | 19.2 | H | 19.20 | H |
| 10 | 20.5 | H | 20.54 | H |
| 11 | 21.8 | H | 21.80 | H |
| 12 | 26.5 | L | 26.48 | L |
| 13 | 27.6 | L | 27.56 | L |

| | | | |
|---|---|---|---|
| normalised intensity values: | L | 0 | 50 |
| | M | 50 | 70 |
| | H | 70 | 100 |

In another embodiment TDCI ULT-1 can be characterised by an XRPD substantially according to FIG. 15A.

The present invention in one aspect relates to a method for the preparation of the crystalline form of Tenofovir Disoproxil citrate TDCI ULT-1 comprising the steps of dissolving or mixing Tenofovir disoproxil free base and citric acid in a suitable solvent or mixture thereof as disclosed herein under ‘Solvents’, preferably chloroform and crystallising Tenofovir Disoproxil citrate TDCI ULT-1 by evaporation of the solvent.

The present invention in another aspect relates to a method for the preparation of the crystalline Tenofovir Disoproxil citrate TDCI ULT-1 comprising the steps of dissolving or mixing Tenofovir disoproxil free base and citric acid in a suitable solvent or mixture thereof as disclosed herein under ‘Solvents’, preferably, and crystallising Tenofovir Disoproxil citrate TDCI ULT-1 by cooling and/or evaporation crystallization of a saturated solution.

The present invention in one aspect relates to a method for the preparation of the crystalline Tenofovir Disoproxil citrate TDCI ULT-1 comprising the steps of dissolving or mixing Tenofovir disoproxil free base and citric acid in a suitable solvent or mixture thereof as disclosed herein under ‘Solvents’, and crystallising Tenofovir Disoproxil citrate TDCI ULT-1 by anti-solvent addition as disclosed herein under ‘Solvents’.

The present invention in another aspect relates to a method for the preparation of the crystalline Tenofovir Disoproxil citrate TDCI ULT-1 comprising the steps of dissolving or mixing Tenofovir disoproxil free base and citric acid in a suitable solvent or mixture thereof as disclosed herein under ‘Solvents’ and crystallising Tenofovir Disoproxil citrate TDCI ULT-1 by slurry crystallisation and/or seed crystallisation.

### Tenofovir Disoproxil Salicylate

Tenofovir Disoproxil Salicylate TDSY ULT-1

Thus, in one aspect, the present invention provides crystalline Tenofovir disoproxil salicylate TDSY ULT-1 herein defined as TDSY ULT-1, characterised by the selection of at least one, preferably at least two, more preferably at least three, even more preferably at least four, particularly preferred at least five and most preferred six X-ray powder diffraction peaks selected from the group consisting of 3.9, 5.1, 6.5, 9.7, 15.2, 16.3, 17.8, 19.0, 21.7, 22.4, 24.0, 27.3 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine X-ray powder diffraction peaks are selected from the above group.

In another embodiment TDSY ULT-1 can be characterised by the following set of XRPD peaks (Table 16) and, optionally, by the associated intensities:

TABLE 16

| | | | Preferred | |
|---|---|---|---|---|
| | Sal1 | | embodiment | |
| Peak ID | Angle (2θ) | Intensity* | Angle (2θ) | Intensity* |
| 1 | 3.9 | H | 3.94 | H |
| 2 | 5.1 | L | 5.06 | L |
| 3 | 6.5 | L | 6.50 | L |
| 4 | 9.7 | L | 9.66 | L |
| 5 | 15.2 | L | 15.18 | L |
| 6 | 16.3 | H | 16.26 | H |
| 7 | 17.8 | L | 17.82 | L |
| 8 | 19.0 | M | 19.02 | M |
| 9 | 21.7 | L | 21.70 | L |
| 10 | 22.4 | M | 22.38 | M |
| 11 | 24.0 | L | 24.02 | L |
| 12 | 27.3 | L | 27.30 | L |

| | | | |
|---|---|---|---|
| normalised intensity values: | L | 0 | 80 |
| | M | 80 | 90 |
| | H | 90 | 100 |

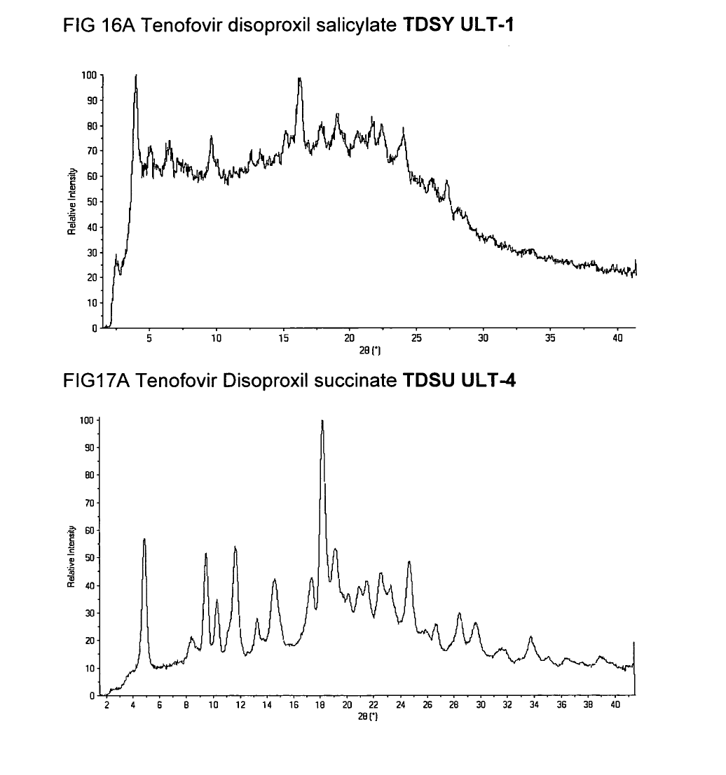

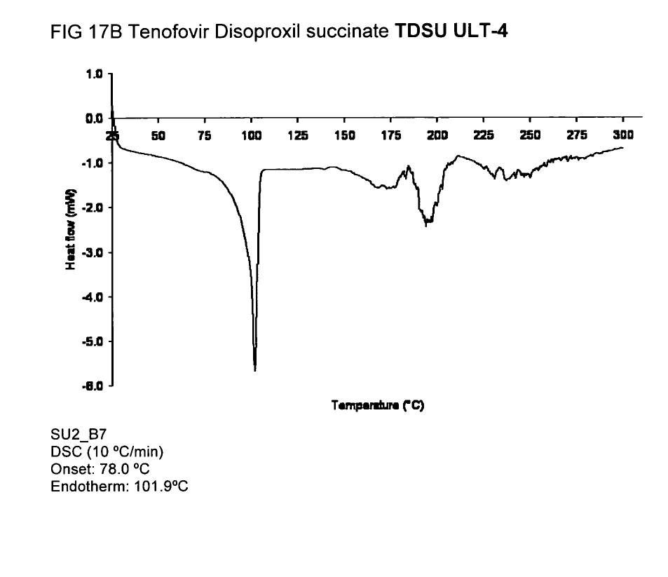

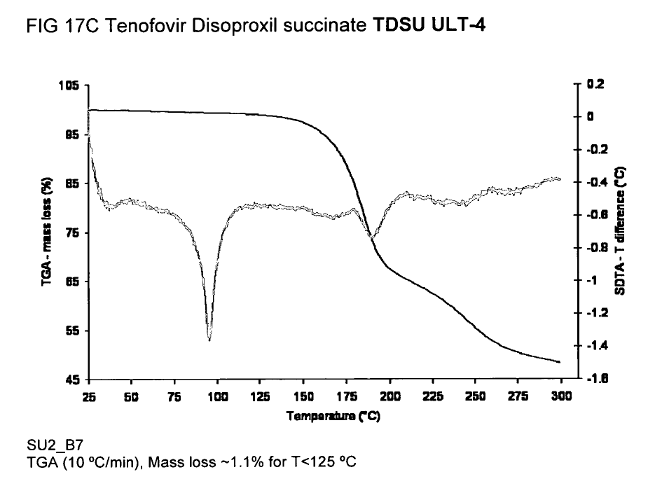

In another embodiment TDSY ULT-1 can be characterised by an XRPD substantially according to FIG. 16A.

The present invention in one aspect relates to a method for the preparation of the crystalline form of Tenofovir Disoproxil salicylate TDSY ULT-1 comprising the steps of dissolving or mixing Tenofovir disoproxil free base and salicylic acid in a suitable solvent or mixture thereof as disclosed herein under ‘Solvents’, preferably acetone, water or mixtures thereof and crystallising Tenofovir Disoproxil salicylate TDSY ULT-1 by evaporation of the solvent.

The present invention in another aspect relates to a method for the preparation of the crystalline Tenofovir Disoproxil salicylate TDSY ULT-1 comprising the steps of dissolving or mixing Tenofovir disoproxil free base and salicylic acid in a suitable solvent or mixture thereof as disclosed herein under ‘Solvents’, preferably acetone, water or mixtures thereof, and crystallising Tenofovir Disoproxil salicylate TDSY ULT-1 by cooling and/or evaporation crystallization of a saturated solution.

The present invention in one aspect relates to a method for the preparation of the crystalline Tenofovir Disoproxil salicylate TDSY ULT-1 comprising the steps of dissolving or mixing Tenofovir disoproxil free base and salicylic acid in a suitable solvent or mixture thereof as disclosed herein under ‘Solvents’, preferably acetone, water or mixtures thereof and crystallising Tenofovir Disoproxil salicylate TDSY ULT-1 by anti-solvent addition as disclosed herein under ‘Solvents’.

The present invention in another aspect relates to a method for the preparation of the crystalline Tenofovir Disoproxil salicylate TDSY ULT-1 comprising the steps of dissolving or mixing Tenofovir disoproxil free base and salicylic acid in a suitable solvent or mixture thereof as disclosed herein under ‘Solvents’, preferably acetone, water or mixtures thereof and crystallising Tenofovir Disoproxil salicylate TDSY ULT-1 by slurry crystallisation and/or seed crystallisation.

Tenofovir Disoproxil Succinate TDSU ULT-4

Thus, in one aspect, the present invention provides crystalline Tenofovir disoproxil succinate, herein defined as TDSU ULT-4 characterised by the selection of at least one, preferably at least two, more preferably at least three, even more preferably at least four, particularly preferred at least five and most preferred six X-ray powder diffraction peaks selected from the group consisting of 4.9, 9.5, 10.3, 11.6, 13.3, 14.5, 17.4, 18.2, 19.2, 24.6, 28.4, 29.6, 33.8 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and, most preferred eleven X-ray powder diffraction peaks are selected from the above group. In a more preferred embodiment, at least twelve, more preferably at least thirteen X-ray powder diffraction peaks are selected from the above group.

In another embodiment TDSU ULT-4 can be characterised by the following set of XRPD peaks (Table 17) and, optionally, by the associated intensities:

TABLE 17

| | | | Preferred | |
|---|---|---|---|---|
| | suc1 | | embodiment | |
| Peak ID | Angle (2θ) | Intensity* | Angle (2θ) | Intensity* |
| 1 | 4.9 | M | 4.86 | M |
| 2 | 9.5 | M | 9.46 | M |
| 3 | 10.3 | L | 10.28 | L |
| 4 | 11.6 | M | 11.64 | M |
| 5 | 13.3 | L | 13.26 | L |
| 6 | 14.5 | L | 14.54 | L |
| 7 | 17.4 | L | 17.36 | L |
| 8 | 18.2 | H | 18.20 | H |
| 9 | 19.2 | M | 19.15 | M |
| 10 | 24.6 | L | 24.64 | L |
| 11 | 28.4 | L | 28.42 | L |
| 12 | 29.6 | L | 29.62 | L |
| 13 | 33.8 | L | 33.75 | L |

| | | | |
|---|---|---|---|
| normalised intensity values: | L | 0 | 50 |
| | M | 50 | 70 |
| | H | 70 | 100 |

In another embodiment, TDSU ULT-4 can be characterised by an XRPD substantially according to FIG. 1A.

In another embodiment, TDSU ULT-4 can be characterised by an DSC substantially according to FIG. 1B.

In another embodiment, TDSU ULT-4 can be characterised by a TGA substantially according to FIG. 1C.

In another embodiment, TDSU ULT-4 of the present invention can be characterised by DSC with an onset at 78.0° C. and a characterising peak at 101.9° C. From the thermal analysis, it is concluded that solid TDSU ULT-4 is anhydrous.

The present invention in one aspect relates to a method for the preparation of the crystalline form of Tenofovir Disoproxil Succinate TDSU ULT-4 comprising the steps of dissolving or mixing Tenofovir disoproxil free base and succinic acid in a suitable solvent or mixture thereof as disclosed herein under ‘Solvents’, preferably methanol and crystallising Tenofovir Disoproxil Succinate TDSU ULT-4 by evaporation of the solvent.

The present invention in another aspect relates to a method for the preparation of the crystalline Tenofovir Disoproxil Succinate TDSU ULT-4 comprising the steps of dissolving or mixing Tenofovir disoproxil free base and succinic acid in a suitable solvent or mixture thereof as disclosed herein under ‘Solvents’, preferably methanol, water or mixtures thereof, and crystallising Tenofovir Disoproxil Succinate TDSU ULT-4 by cooling and/or evaporation crystallization of a saturated solution.

The present invention in one aspect relates to a method for the preparation of the crystalline Tenofovir Disoproxil Succinate TDSU ULT-4 comprising the steps of dissolving or mixing Tenofovir disoproxil free base and succinic acid in a suitable solvent or mixture thereof as disclosed herein under ‘Solvents’, and crystallising Tenofovir. Disoproxil Succinate TDSU ULT-4 by anti-solvent addition as disclosed herein under ‘Solvents’.

The present invention in another aspect relates to a method for the preparation of the crystalline Tenofovir Disoproxil Succinate TDSU ULT-4 comprising the steps of dissolving or mixing Tenofovir disoproxil free base and succinic acid in a suitable solvent or mixture thereof as disclosed herein under ‘Solvents’ and crystallising Tenofovir Disoproxil Succinate TDSU ULT-4 by slurry crystallisation and/or seed crystallisation.

Purity

In one aspect of the invention, all the above forms of tenofovir disoproxil of the present invention are, independently, in a substantially pure form, preferably substantially free from other amorphous, and/or crystalline solid forms. In this respect, “substantially pure” relates to at least about 80%, 85%, 90%, 95%, 96%, 97%, 98%, or 99% of the pure compound. In this respect, “substantially free from other amorphous, and/or crystalline solid forms” means that no more than about 20%, 15%, 10%, 5%, 4%, 3%, 2%, 1% of these other amorphous, and/or crystalline solid forms are present in the form according to the invention.

Solvents

In certain embodiments of the method for the preparation of the forms of the present invention, the solvents for evaporation crystallisation, hot filtration anti-solvent addition, seed crystallisation and/or slurry crystallisation are preferably selected from the group consisting of: (R)-(−)-2-octanol, 1,2-diethoxyethane, 1,2-dimethoxyethane, 1,4-dioxane, 1-butanol, 1-heptanol, 1-hexanol, 1-methoxy-2-propanol, 1-nitropropane, 1-octanol, 2,2,2-trifluoroethanol, 2-butanone, 2-ethoxyethanol, 2-ethoxyethyl acetate, 2-hexanol, 2-methoxyethanol, 2-nitropropane, 2-pentanol, 2-propanol, 4-hydroxy-4-methyl-2-pentanon, acetone, acetonitrile, butyronitrile, cyclohexanol, cyclopentanol, cyclopentanone, diethylene glycol dimethylether, dimethylcarbonate, dimethylcarbonate, ethanol, ethyl formate, ethylacetate, ethylene glycol monobutyl ether, furfuryl alcohol, isobutanol, isopropyl acetate, methanol, methoxyethyl acetate, methyl acetate, methyl butyrate, methyl propionate, methyl-4-2-pentanol, n,n-dimethylacetamide, n,n-dimethylformamide, nitrobenzene, nitroethane, nitromethane, n-methylpyrrolidone, propionitrile, propyl acetate, propylene glycol methyl ether acetate, tert-butanol, tetrahydrofuran, tetrahydrofurfurylalcohol, tetrahydropyran, water or mixtures thereof.

In certain embodiments of the method for the preparation of the solid forms of Tenofovir Disoproxil of the present invention, the solvents for hot filtration crystallisation are preferably selected from the group consisting of: (R)-(−)-2-Octanol, 1,2-Diethoxyethane, 1,2-Dimethoxyethane, 1,4-Dioxane, 1-Butanol, 1-Nitropropane, 1-Propanol, 2-Butanone, 2-Ethoxyethyl acetate, 2-Methyl-4-pentanol, 2-Nitropropane, 2-Propanol, Acetone, Acetonitrile, Cyclopentanol, Ethanol, Isobutanol, Isopropyl acetate, Methanol, Methoxy-2-1-Propanol, Methyl propionate, N,N-Dimethylacetamide, N,N-Dimethylformamide, Nitromethane, tert-Butanol, Tetrahydrofuran, Water or mixtures thereof.

In certain embodiments of the method for the preparation of the solid forms of the present invention, the solvents for solvent/anti-solvent crystallisation are preferably selected from the group consisting of: 1,2-Dichloroethane, 1,2-Dimethoxyethane, 1,4-Dioxane, 2,6-Dimethyl-4-heptanone, 2-Butanone, Acetone, Acetonitrile, Amyl ether, Butyl benzene, Chloroform, Cyclohexane, Cyclohexane, Cyclohexane, Cyclohexane, Cyclohexane, Dichloromethane, Hexafluorobenzene, Methanol, n-Heptane, Nitromethane, N-Methyl Pyrrolidone, tert-Butyl methyl ether, Tetrahydrofuran, Toluene; Water or mixtures thereof.

In certain embodiments of the method for the forms of the present invention, the anti-solvents for anti-solvent crystallisation are preferably selected from the group consisting of: 1,2-Dichloroethane, 2,6-Dimethyl-4-heptanone, Acetone, Amyl ether, Butyl benzene, Chloroform, Cyclohexane, Dichloromethane, Hexafluorobenzene, n-Heptane, Nitromethane, tert-Butyl methyl ether, Toluene or mixtures thereof.

In certain embodiments of the method for the preparation of the forms of the present invention, the solvents for seeding crystallisation are preferably selected from the group consisting of: methanol, water, 1,4-dioxane, acetonitrile, 2-ethoxyethylacetate, 2-methyl-4-pentanol, tetrahydrofuran, butyl benzene, amylether, tert-butyl methyl ether, cyclopentanone or mixtures thereof.

In certain embodiments of the method for the preparation of the forms of the present invention, the solvents for slurrying crystallisation are preferably selected from the group consisting of: water, methanol, acetonitrile, 1,4-dioxane or mixtures thereof.

Pharmaceutical Formulations.

The present invention further relates to pharmaceutical formulations comprising the novel crystalline forms of Tenofovir DF.

Pharmaceutical formulations of the present invention contain one or more of the crystalline forms according to the present invention, as disclosed herein. The invention also provides pharmaceutical compositions comprising one or more of the crystal forms according to the present invention. Pharmaceutical formulations of the present invention contains one or more of the crystal form according to the present invention as active ingredient, optionally in a mixture with other crystal form(s).

The pharmaceutical formulations according to the invention, may further comprise, in addition to the solid forms described herein additional pharmaceutical active ingredients, preferably Anti-HIV agents and more preferably Efavirenz and/or Emtricitabine.

In addition to the active ingredient(s), the pharmaceutical formulations of the present invention may contain one or more excipients. Excipients are added to the formulation for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), micro fine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Solid and liquid compositions may also be dyed using any pharmaceutically acceptable colorant to improve their appearance and/or facilitate patient identification of the product and unit dosage level.

In liquid pharmaceutical compositions of the present invention, the crystalline forms according to the present invention and any other solid excipients are suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methylcellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxyl toluene, butylated hydroxyanisole and ethylenediamine tetraacetic acid may be added at levels safe for ingestion to improve storage stability. According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

For infections of the eye or other external tissues, e.g. mouth and skin, the formulations are preferably applied as a topical ointment or cream containing the active ingredient(s) in an amount of, for example, 0.01 to 10% w/w (including active ingredient(s) in a range between 0.1% and 5% in increments of 0.1% w/w such as 0.6% w/w, 0.7% w/w, etc), preferably 0.2 to 3% w/w and most preferably 0.5 to 2% w/w. When formulated in an ointment, the active ingredients may be employed with either a paraffinic or a water-miscible ointment base.

Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) or mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the emulsifying wax, and the wax together with the oil and fat make up the emulsifying ointment base which forms the oily dispersed phase of the cream formulations.

Emulgents and emulsion stabilisers suitable for use in the formulation of the present invention include Tween8 60, Spans 80, cetostearyl alcohol, benzyl alcohol, myristyl alcohol, glyceryl monostearate and sodium lauryl sulfate.

The choice of suitable oils or fats for the formulation is based on achieving the desired cosmetic properties. Thus the cream should preferably be a non-greasy, non-staining and washable product with suitable consistency to avoid leakage from tubes or other containers.

Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate,2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is suitably present in such formulations in a concentration of 0.01 to 20%, in some embodiments 0.1 to 10%, and in others about 1.0% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal or inhalational administration wherein the carrier is a solid include a powder having a particle size for example in the range 1 to 500 microns (including particle sizes in a range between 20 and 500 microns in increments of 5 microns such as 30 microns, 35 microns, etc). Suitable formulations wherein the carrier is a liquid, for administration as for example a nasal spray or as nasal drops, include aqueous or oily solutions of the active ingredient.

Formulations suitable for aerosol administration may be prepared according to conventional methods and may be delivered with other therapeutic agents. Inhalational therapy is readily administered by metered dose inhalers.

Formulations suitable for vaginal administration may be presented as pessaries, tampons, creams, gels, pastes, foams or spray formulations containing in addition to the active ingredient such carriers as are known in the art to be appropriate.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art. A composition for tabletting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tabletted/compressed, or other excipients may be added prior to tabletting, such as a glidant and/or a lubricant.

A tabletting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Moreover, the crystalline forms according to the present invention can be formulated for administration to a mammal, preferably a human, via injection. The crystalline forms according to the present invention may be formulated, for example, as a viscous liquid solution or suspension, preferably a clear solution, for injection. The formulation may contain solvents. Among considerations for such solvent include the solvent's physical and chemical stability at various pH levels, viscosity (which would allow for syringeability), fluidity, boiling point, miscibility and purity. Suitable solvents include alcohol USP, benzyl alcohol NF, benzyl benzoate USP and Castor oil USP. Additional substances may be added to the formulation such as buffers, solubilizers, antioxidants, among others. Ansel et al., Pharmaceutical Dosage Forms and Drug Delivery Systems, 7th Ed.

The present invention also provides pharmaceutical formulations comprising the crystalline form according to the present invention, optionally in combination with other polymorphic forms or co-crystals, to be used in a method of treatment of a mammal, preferably a human, in need thereof. A pharmaceutical composition of the present invention comprises the crystalline form. The crystalline form according to the present invention may be used in a method of treatment of a mammal comprising administering to a mammal suffering from the ailments described herein before a therapeutically effective amount of such pharmaceutical composition. The invention further relates to the use of the crystalline form of the invention for the preparation of a medicament for the treatment of the ailments described herein before, in particular HIV.

Having described the invention with reference to certain preferred embodiments, other embodiments will become apparent to one skilled in the art from consideration of the specification. The invention is further defined by reference to the following examples describing in detail the preparation of the compounds of the present invention. It will be apparent to those skilled in the art that many modifications, both to materials and methods, may be practiced without departing from the scope of the invention.

### EXAMPLES

### Experimental Conditions

X-ray Powder Diffraction:

XRPD patterns were obtained using a T2 high-throughput XRPD set-up by Avantium technologies, The Netherlands. The plates were mounted on a Bruker GADDS diffractometer equipped with a Hi-Star area detector. The XRPD platform was calibrated using Silver Behenate for the long d-spacings and Corundum for the short d-spacings. Data collection was carried out at room temperature using monochromatic CuK(alpha)radiation in the two-theta region between 1.5° and 41.5°. The diffraction pattern of each well is collected in two two-theta ranges (1.5°≦2θ21.5° for the first frame, and 19.5° 2θ≦41.5° for the second) with an exposure time of 120 s for each frame. One of ordinary skill in the art understands that experimental differences may arise due to differences in instrumentation, sample preparation, or other factors. Typically XRPD data are collected with a variance of about 0.3 degrees two-theta, preferable about 0.2 degrees, more preferably 0.1 degrees, even more preferable 0.05 degrees. This has consequences for when X-ray peaks are considered overlapping.

Thermal Analysis:

Melting properties were obtained from DSC thermograms, recorded with a heat flux DSC822e instrument (Mettler-Toledo GmbH, Switzerland). The DSC822e was calibrated for temperature and enthalpy with a small piece of indium (m.p.=156.6° C.; delta-H(f)=28.45 J/g). Samples were sealed in standard 40 microliter aluminum pans and heated in the DSC from 25° C. to 300° C., at a heating rate of 20° C./min. Dry N₂ gas, at a flow rate of 50 ml/min, was used to purge the DSC equipment during measurement.

Mass loss due to solvent or water loss from the crystals was determined by TGA/SDTA. Monitoring of the sample weight, during heating in a TGA/SDTA851e instrument (Mettler-Toledo GmbH, Switzerland), resulted in a weight vs. temperature curve. The TGA/SDTA851e was calibrated for temperature with indium and aluminium. Samples were weighed into 100 microliter aluminium crucibles and sealed. The seals were pin-holed and the crucibles heated in the TGA from 25° C. to 300° C. at a heating rate of 20° C./min. Dry N₂ gas is used for purging. Melting point determinations based on DSC have a variability of +/−2.0 degrees Celsius, preferably 1.0 degrees Celsius.

Dynamic Vapour Sorption (DVS)

Moisture sorption isotherms were measured using a DVS-1 system of Surface Measurement Systems (London, UK). Differences in moisture uptake of a solid material indicate differences in the relative stabilities of the various solid forms for increasing relative humidity. The experiment was carried out at a constant temperature of 25° C.

### Examples

The starting material for the crystallisation experiments was obtained as a research sample from Cipla Ltd, Mumbai, India and converted to the fee base using common procedures

### Crystallization of Solid Forms at Microliter Scale.

A small quantity, about 3 mg of the starting material was stock dosed in a each of the wells of a 96-well plate using in 1,4-dioxane as stock solvent. The plates were placed under vacuum until the solvent evaporated. Following, the counter ions were added in each well at a counter-ion:free-base ratio of 1.1:1, either by solid dosing or by a stock solution in 1,4-dioxane or water. In the cases that the counter ion was dosed by means of a stock solution, the solvent was removed by evaporation. Subsequently, 30 μL of a crystallization solvent was added and the plates were heated to 60° C. for 60 min. The solutions were cooled with 1.1° C./h to a temperature of 5 or 20° C. where they remained for 24 h. Subsequently, the solvents were evaporated from the wells under 20 kPa pressure at 20-25° C. for 19-24 h. The resulting residue was harvested and analyzed by X-ray powder diffraction.

The counter ions used and the corresponding crystallization solvents are listed in Table 1.

TABLE 1

| Form | Counter ion | Crystallization Solvent | T end (° C.) |
|---|---|---|---|
| TDTA ULT-2 | Tartaric acid, L- | Acetonitrile | 5 |
| TDTA ULT-3 | Tartaric acid, L- | Acetone | 20 |
| TDOX ULT-1 | oxalic acid | Chloroform | 20 |
| TDOX ULT-1 | Oxalic acid | Chloroform | 5 |
| TDOX ULT-1 | oxalic acid | Acetonitrile/water | 20 |
| | | (50/50) | |
| TDOX ULT-1 | Oxalic acid | Acetone | 5 |
| TDOX ULT-1 | oxalic acid | Methanol | 20 |
| TDOX ULT-1 | Oxalic acid | Acetonitrile | 5 |
| TDOX ULT-1 | Oxalic acid | Tetrahydrofuran | 5 |
| TDOX ULT-3 | oxalic acid | Dioxane 1,4- | 20 |
| TDOX ULT-3 | Oxalic acid | Water | 5 |
| TDOX ULT-3 | oxalic acid | Acetone | 20 |
| TDOX ULT-3 | oxalic acid | Acetone | 20 |
| TDSA ULT-1 | Saccharin | Nitromethane | 5 |
| TDSA ULT-2 | Saccharin | Chloroform | 20 |
| TDSA ULT-2 | Saccharin | Chloroform | 5 |
| TDSY ULT-1 | Salicylic acid | Acetone/Water (50/50) | 20 |
| TDSY ULT-1 | Salicylic acid | Water | 5 |
| TDSY ULT-1 | Salicylic acid | Acetone/Water (50/50) | 5 |
| TDSY ULT-1 | Salicylic acid | Water | 5 |
| TDSU ULT-1 | Succinic acid | Methanol/water | 5 |
| TDSU ULT-2 | Succinic acid | Chloroform | 5 |
| TDSU ULT-3 | Succinic acid | Acetone | 5 |
| TDSU ULT-4 | Succinic acid | Methanol | 5 |

Crystallization of Solid Forms at Milliliter Scale.

About 50 mg of the free base was solid dosed into vials together with the counter ions at a counter-ion:free-base ratio of 1.1:1. The crystallization solvents were added so that the concentration with respect to the free base was 100 mg/ml. The vials were heated to 60° C. for 60 min. The solutions were cooled with 1.1° C./h to a temperature of 5 or 20° C. where they remained for 24 h. In the cases in which solids precipitated after ageing, the solid material was separated by centrifugation, dried and measured by XRPD. The supernatant solution of each separation was also evaporated under 20 kPa pressure at 20-25° C. for 70-170 h and the dried solids were also measured by XRPD.

The counter ions used and the corresponding crystallization solvents are listed in Table 2.

TABLE 2

| Form | Counter ion | Crystallisation Solvent | T end (° C.) |
|---|---|---|---|
| TDSU ULT-1 | Succinic acid | methanol | 5 |
| TDSU ULT-1 | Succinic acid | acetonitrile, | 5 |
| TDSU ULT-1 | Succinic acid | acetone | 5 |
| TDSU ULT-2 | Succinic acid | Dioxane 1,4- | 5 |
| TDSU ULT-3 | Succinic acid | Acetone | 5 |
| TDSU ULT-4 | Succinic acid | Methanol | 20 |
| TDSU ULT-4 | Succinic acid | Methanol/Water | 5 |
| TDTA ULT-1 | L-Tartaric acid | Chloroform | 20 |
| TDTA ULT-1 | L-Tartaric acid | Chloroform | 5 |
| TDTA ULT-3 | L-Tartaric acid | Tetrahydrofuran | 20 |
| TDTA ULT-3 | L-Tartaric acid | Acetone | 5 |
| TDTA ULT-4 | L-Tartaric acid | Acetonitrile | 5 |
| TDOX ULT-2 | Oxalic acid | Acetone/Water | 5 |
| TDOX ULT-2 | Oxalic acid | Acetone | 5 |
| TDOX ULT-2 | Oxalic acid | Acetonitrile | 5 |
| TDOX ULT-3 | Oxalic acid | Water | 5 |
| TDOX ULT-4 | Oxalic acid | Methanol/Water | 5 |
| TDSA ULT-3 | Saccharin | Chloroform | 20 |
| TDSY ULT-1 | Salicylic acid | Acetone/Water | 20 |
| TDCI ULT-1 | Citric acid | Chloroform | 5 |

TDSU ULT-1

About 997 mg of tenofovir disoproxil free base was placed in a 50 ml glass reactor together with succinic acid at about 1.1:1 counter-ion:free-base molecular ratio. The crystallization solvents were added so that the concentration with respect to the free base was 100 mg/ml. The reactor was heated to 60° C. with a heating rate of 5° C./min and maintain at 60° C. for 60 min. Subsequently, the solutions were cooled with 1.1° C./h to a temperature of 5 where they remained for 24 h. At the end the solutions were filtered by using Buckner Filter with 0.5 micron filter mesh, dried at room temperature under vacuum and measured by XRPD.

The solvents Methanol and Acetonitrile were used as crystallization solvents.

A sample of about 15 mg of TDSU ULT-1 was spread in the DVS pan. The sample was dried at 0% RH for 7 h. Subsequently the relative humidity of the chamber was increased in steps of 5% units from 0% to 95% in order to monitor the sorption of water vapours. The samples remained in each of the steps for 1 h. Following, desorption was monitored by decreasing the relative humidity to 0% in steps of 5% units and remaining at each step for 1 h. The graph of sorption-desorption cycle is shown below. The total uptake of water vapours was about 0.3% demonstrating good stability of the material and no hygroscopicity which is line with the industry standard for hygroscopicity. In a similar DVS experiment using the starting material as purchased, the total vapour intake was about 4%, which is undesirable in formulation and requires additional measures. At the end of the experiment, the solid material was measured by XRPD which showed that there were no any changes in the structure.

Dissolution Rate Measurements

20 ml of high pure water was placed in 25 ml vial in the micro-dissolution thermal block by using a 20 ml volumetric pipette. A large cross stirrer was placed to the vial and the solution was stirred at a speed of 100 rpm. The 5 mm path length tip was placed from the top along with the probe connected with DAD (Diode Array Detector) analyzer. The 100% transmittance and dark spectra was collected by using high pure water. In the next step a tablet of 10 mg of Tenofovir disoproxil succinate (TDSU ULT-1) was pressed on tablet machine and placed along with the stirrer in a 25 ml vial in the micro-dissolution thermal block. The probe was placed along with the 5 mm path length tip and 20 ml of high pure water was added to the sample. The solution was stirred with a speed of 100 rpm and absorbance or the optical density was determined with respect to time by UV spectrometer. The intrinsic dissolution rate was determined by plotting concentration versus time and calculating the slope of the curve.

The same experiments were performed in a similar manner in buffered media of pH values of 1.5, 3.0, 4.5, 6.4 and 7.8.

pH buffers for dissolution

pH 1.5: USP SGF without pepsin (0.05M sodium chloride adjusted to pH 1.5 with HCl)

pH 3.0: 0.05 sodium di-hydrogen phosphate buffer adjusted to pH 6.8 with NaOH

pH 4.5: 0.05M sodium di-hydrogen phosphate buffer adjusted to pH 4.5 with NaOH

pH 6.8: USP SIF without pancreatin (0.05M sodium di-hydrogen phosphate buffer adjusted to pH 6.8 with NaOH)

pH 7.4: 0.05M sodium di-hydrogen phosphate adjusted to pH 7.4 with NaOH

Results of intrinsic dissolution rate measurements

| medium | Dissolution rate (mgmin−1 cm−2) | TDF 1:1 |
|---|---|---|
| Buffer pH 1.5 | 10.00 | 3.29 |
| Buffer pH 3.0 | 1.78 | 1.36 |
| Buffer pH 4.5 | 3.55 | 0.99 |
| Buffer pH 6.8 | 1.88 | 0.98 |
| Buffer pH 7.4 | 1.09 | |
| water | 1.42 | 1.12 |

A small quantity (of about 10 milligrams) of TDSU ULT-1 was placed in a Binder climatic chamber KBR115 series for stress testing under 40° C. and 75% relative humidity (RH). The material was checked for physical and chemical stability by XRPD and HPLC respectively in intervals of 2 and 4 weeks. In both cases the material was stable, i.e. no structural change or chemical degradation took place over the tested period.

From the DVS data it was concluded that TDSU ULT-1 is not hygroscopic (max water adsorption of about 0.3%—which is considerably less hygroscopic than TDF 1:1 obtained from Cipla (hygroscopicity of about 4%).

TDSU ULT-1 is dissolving up to 3 times faster compared to the tenofovir fumarate (TDF 1:1 obtainable from Cipla) in water and in all media with pH values ranging from 1.5 to 7.4.

TDSU ULT-4 (SU39)

About 53 mg of the free base was solid dosed into a vial together with 13.15 mg of succinic acid. Methanol was added so that the concentration with respect to the free base was 100 mg/ml. The vial was heated to 60° C. for 60 min. The solution was cooled with 1.1° C./h to a temperature of 5° C. where it remained for 24 h. The solvent was removed by evaporation under 20 kPa pressure at 20-25° C. and the dried solids were measured by XRPD.

TDTA ULT-1 (SU27)

About 50.6 mg of the free base was solid dosed into a vial together with 18.73 mg of L-tartaric acid. Chloroform was added so that the concentration with respect to the free base was 100 mg/ml. The vial was heated to 60° C. for 60 min. The solution was cooled with 1.1° C./h to a temperature of 5° C. where it remained for 24 h. The solvent was removed by evaporation under 20 kPa pressure at 20-25° C. and the dried solids were measured by XRPD.

TDOX ULT-3 (SU35)

About 53.7 mg of the free base was solid dosed into a vial together with 10.36 mg of oxalic acid. Water was added so that the concentration with respect to the free base was 100 mg/ml. The vial was heated to 60° C. for 60 min. The solution was cooled with 1.1° C./h to a temperature of 5° C. where it remained for 24 h. The precipitated solids were separated by centrifugation and the solids were dried and measured by XRPD. The supernatant solution was evaporated under 20 kPa pressure at 20-25° C. and the dried solids were also measured by XRPD. In both cases XRPD indicated the solid form Oxa2.

TDSA ULT-3 (SU36)

About 53.7 mg of the free base was solid dosed into a vial together with 22.5 mg of saccharin. Chloroform was added so that the concentration with respect to the free base was 100 mg/ml. The vial was heated to 60° C. for 60 min. The solution was cooled with 1.1° C./h to a temperature of 20° C. where it remained for 24 h. The solvent was removed by evaporation under 20 kPa pressure at 20-25° C. and the dried solids were measured by XRPD.

1.-62. (canceled)

63. A solid form of Tenofovir disoproxil and an organic acid selected from the group consisting of succinic acid, tartaric acid, saccharic acid, citric acid, salicylic acid.

64. The solid form of Tenofovir disoproxil and an organic acid according to claim 63, selected from the group consisting of Tenofovir disoproxil succinate, Tenofovir disoproxil L-tartrate, Tenofovir disoproxil oxalate, Tenofovir disoproxil saccharate, Tenofovir disoproxil citrate, Tenofovir disoproxil salicylate.

65. The solid form of Tenofovir disoproxil and an organic acid according to claim 63, selected from the group consisting of Tenofovir disoproxil succinate, Tenofovir disoproxil L-tartrate, Tenofovir disoproxil oxalate, Tenofovir disoproxil saccharate, Tenofovir disoproxil citrate, Tenofovir disoproxil salicylate wherein the solid form is crystalline.

66. The solid form of Tenofovir disoproxil and an organic acid according to claim 63, selected from the group consisting of Tenofovir disoproxil succinate TDSU ULT-1, Tenofovir disoproxil succinate TDSU ULT-2, Tenofovir disoproxil succinate TDSU ULT-3, Tenofovir disoproxil succinate TDSU ULT-4 Tenofovir disoproxil L-tartrate TDTA ULT-1, Tenofovir disoproxil L-tartrate TDTA ULT-2, Tenofovir disoproxil L-tartrate TDTA ULT-3, Tenofovir disoproxil L-tartrate TDTA ULT-4, Tenofovir disoproxil oxalate TDOX ULT-1, Tenofovir disoproxil oxalate TDOX ULT-2, Tenofovir disoproxil oxalate TDOX ULT-3, Tenofovir disoproxil oxalate TDOX ULT-4, Tenofovir disoproxil saccharate TDSA ULT-1, Tenofovir disoproxil saccharate TDSA ULT-2 Tenofovir disoproxil saccharate TDSA ULT-3, Tenofovir disoproxil citrate TDCI ULT-1, Tenofovir disoproxil salicylate TDSY ULT-1.

67. The solid form of Tenofovir disoproxil and an organic acid according to claim 63, which is Tenofovir disoproxil succinate TDSU ULT-1, wherein:
at least one, preferably at least two, more preferably at least three, even more preferably at least four, particularly preferred at least five and most preferred six X-ray powder diffraction peaks selected from the group consisting of 4.9, 9.5, 10.3, 11.5, 13.3, 14.7, 17.9, 18.2, 19.1, 24.7, 29.8 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 102.0° C. and a characterising peak at 111.0° C.

68. The solid form of Tenofovir disoproxil and an organic acid according to claim 63, which is Tenofovir disoproxil succinate TDSU ULT-1, wherein:
a XRPD pattern substantially as set out in Table 1 and/or FIG. 1A;
a DSC substantially as set out in FIG. 1B; and/or
a TGA substantially as set out in FIG. 1C.

69. A method for the preparation of the form of Tenofovir disoproxil succinate TDSU ULT-1 comprising the steps of
dissolving or mixing Tenofovir disoproxil free base and succinic acid in a suitable solvent or mixture thereof, preferably methanol, ether, acetone, acetonitrile or mixtures thereof (such as 50/50 v/v methanol-ether) and crystallising Tenofovir Disoproxil succinate TDSU ULT-1 by evaporation of the solvent; and/or
dissolving or mixing Tenofovir disoproxil free base and succinic acid in a suitable solvent or mixture thereof, preferably methanol, ether, acetone, acetonitrile or mixtures thereof (such as 50/50 v/v methanol-ether) and crystallising Tenofovir Disoproxil succinate TDSU ULT-1 by cooling and/or evaporation crystallization of a saturated solution; and/or
dissolving or mixing Tenofovir disoproxil free base and succinic acid in a suitable solvent or mixture thereof and crystallising Tenofovir Disoproxil succinate TDSU ULT-1 by anti-solvent addition; and/or
dissolving or mixing Tenofovir disoproxil free base and succinic acid in a suitable solvent or mixture thereof and crystallising Tenofovir Disoproxil succinate TDSU ULT-1 by slurry crystallisation and/or seed crystallisation.

70. The solid form of Tenofovir disoproxil and an organic acid according to claim 63, which is Tenofovir disoproxil succinate TDSU ULT-2, wherein:
at least one, preferably at least two, more preferably at least three, even more preferably at least four, particularly preferred at least five and most preferred six X-ray powder diffraction peaks selected from the group consisting of 4.8, 6.6, 9.5, 10.6, 12.6, 13.4, 17.2, 18.4, 19.0, 21.3, 24.1 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 92.6° C. and a characterising peak at 107.7° C.

71. The solid form of Tenofovir disoproxil and an organic acid according to claim 63, which is Tenofovir disoproxil succinate TDSU ULT-3, wherein:
at least one, preferably at least two, more preferably at least three, even more preferably at least four, particularly preferred at least five and most preferred six X-ray powder diffraction peaks selected from the group consisting of 4.8, 9.5, 10.3, 11.0, 11.7, 13.2, 14.0, 17.1, 18.2, 19.1, 23.3, 23.6 degrees two-theta+/−0.3 degrees two-theta, preferably +/−0.2 degrees two-theta, more preferably +/−0.1 degrees two-theta, most preferably +/−0.05 degrees two-theta.

72. The solid form of Tenofovir disoproxil and an organic acid according to claim 63, which is Tenofovir disoproxil succinate TDSU ULT-4, wherein:
at least one, preferably at least two, more preferably at least three, even more preferably at least four, particularly preferred at least five and most preferred six X-ray powder diffraction peaks selected from the group consisting of 4.9, 9.5, 10.3, 11.6, 13.3, 14.5, 17.4, 18.2, 19.2, 24.6, 28.4, 29.6, 33.8 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 78.0° C. and a characterising peak at 101.9° C.

73. The solid form of Tenofovir disoproxil and an organic acid according to claim 63, which is Tenofovir disoproxil tartrate TDTA ULT-1, wherein:
at least one, preferably at least two, more preferably at least three, even more preferably at least four, particularly preferred at least five and most preferred six X-ray powder diffraction peaks selected from the group consisting of 4.9, 8.8, 9.6, 12.8, 13.5, 14.6, 16.2, 18.9, 20.8, 21.5, 22.3 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset a 7.91° C. and a characterising peak at 98.1° C.

74. The solid form of Tenofovir disoproxil and an organic acid according to claim 63, which is Tenofovir disoproxil tartrate TDTA ULT-2, wherein:
at least one, preferably at least two, more preferably at least three, even more preferably at least four, particularly preferred at least five and most preferred six X-ray powder diffraction peaks selected from the group consisting of 5.2, 7.8, 8.8, 9.1, 10.4, 11.8, 12.9, 13.7, 14.8, 15.9, 16.4, 18.2, 20.4, 21.2, 22.4, 24.0 degrees two-theta+/−0.3 degrees two-theta, preferably +/−0.2 degrees two-theta, more preferably +/−0.1 degrees two-theta, most preferably +/−0.05 degrees two-theta.

75. The solid form of Tenofovir disoproxil and an organic acid according to claim 63, which is Tenofovir disoproxil tartrate TDTA ULT-3, wherein:
at least one, preferably at least two, more preferably at least three, even more preferably at least four, particularly preferred at least five and most preferred six X-ray powder diffraction peaks selected from the group consisting of 4.9, 9.0, 11.9, 13.0, 13.8, 15.0, 17.9, 19.3, 20.08, 21, 21.6, 22.5, 23.1, 23.6, 26.5, 28.3 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 80° C. and a characterising peak at 105° C.

76. The solid form of Tenofovir disoproxil and an organic acid according to claim 63, which is Tenofovir disoproxil tartrate TDTA ULT-4, wherein:
at least one, preferably at least two, more preferably at least three, even more preferably at least four, particularly preferred at least five and most preferred six X-ray powder diffraction peaks selected from the group consisting of 5.1, 8.9, 10.0, 12.7, 13.7, 14.7, 15.7, 17.7, 20.0, 20.9, 21.6, 25.4 degrees two-theta+/−0.3 degrees two-theta, preferably +/−0.2 degrees two-theta, more preferably +/−0.1 degrees two-theta, most preferably +/−0.05 degrees two-theta.

77. The solid form of Tenofovir disoproxil and an organic acid according to claim 63, which is Tenofovir disoproxil oxalate TDOX ULT-1, wherein:
at least one, preferably at least two, more preferably at least three, even more preferably at least four, particularly preferred at least five and most preferred six X-ray powder diffraction peaks selected from the group consisting of 3.8, 7.6, 9.3, 15.0, 16.4, 17.7, 19.6, 22.6 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 48.0° C. and a characterising peak at 64.8° C., with an onset at 112.6 and a characterising peak at 118.6° C., and/or with an onset at 130.7° C. and a characterising peak at 148.2° C.

78. Solid form of Tenofovir disoproxil and an organic acid according to claim 63, which is Tenofovir disoproxil oxalate TDOX ULT-2, wherein:
at least one, preferably at least two, more preferably at least three, even more preferably at least four, particularly preferred at least five and most preferred six X-ray powder diffraction peaks selected from the group consisting of 3.8, 7.6, 9.3, 15.0, 16.4, 17.7, 19.6, 22.6 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 106.0° C. and a characterising peak at 117.1°9.

79. The solid form of Tenofovir disoproxil and an organic acid according to claim 63, which is Tenofovir disoproxil oxalate TDOX ULT-3, wherein:
at least one, preferably at least two, more preferably at least three, even more preferably at least four, particularly preferred at least five and most preferred six X-ray powder diffraction peaks selected from the group consisting of 3.9, 7.7, 9.4, 16.1, 16.8, 17.5, 18.8, 19.7, 21.6, 22.4, 24.0, 28.1 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 78.4° C. and a characterising peak at 90.9° C.

80. The solid form of Tenofovir disoproxil and an organic acid according to claim 63, which is Tenofovir disoproxil oxalate TDOX ULT-4, wherein:
at least one, preferably at least two, more preferably at least three, even more preferably at least four, particularly preferred at least five and most preferred six X-ray powder diffraction peaks selected from the group consisting of 3.9, 7.8, 8.5, 9.6, 10.9, 15.7, 17.1, 18.8, 20.4, 23.6 degrees two-theta+/−0.3 degrees two-theta, preferably +/−0.2 degrees two-theta, more preferably +/−0.1 degrees two-theta, most preferably +/−0.05 degrees two-theta.

81. The solid form of Tenofovir disoproxil and an organic acid according to claim 63, which is Tenofovir disoproxil saccharate TDSA ULT-1, wherein:
at least one, preferably at least two, more preferably at least three, even more preferably at least four, particularly preferred at least five and most preferred six X-ray powder diffraction peaks selected from the group consisting of 3.3, 4.1, 7.6, 10.4, 13, 13.6, 17.9, 18.7, 22.7 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 95.0° C. and a characterising peak at 116.0° C.

82. The solid form of Tenofovir disoproxil and an organic acid according to claim 63, which is Tenofovir disoproxil saccharate TDSA ULT-2, wherein:
at least one, preferably at least two, more preferably at least three, even more preferably at least four, particularly preferred at least five and most preferred six X-ray powder diffraction peaks selected from the group consisting of 3.4, 6.2, 15.3, 15.6, 16.2, 19.7, 22.4, 24.4 degrees two-theta+/−0.3 degrees two-theta, preferably +/−0.2 degrees two-theta, more preferably +/−0.1 degrees two-theta, most preferably +/−0.05 degrees two-theta.

83. The solid form of Tenofovir disoproxil and an organic acid according to claim 63, which is Tenofovir disoproxil saccharate TDSA ULT-3, wherein:
at least one, preferably at least two, more preferably at least three, even more preferably at least four, particularly preferred at least five and most preferred six X-ray powder diffraction peaks selected from the group consisting of 3.94, 7.57, 10.42, 12.58, 15.34, 16.46, 17.68, 20.46, 21.94, 24.66 degrees two-theta+/−0.3 degrees two-theta, preferably +/−0.2 degrees two-theta, more preferably +/−0.1 degrees two-theta, most preferably +/−0.05 degrees two-theta; and/or
DSC with an onset at 68.0° C. and a characterising peak at 83.9° C.

84. The solid form of Tenofovir disoproxil and an organic acid according to claim 63, which is Tenofovir disoproxil citrate TDCI ULT-1, wherein:
at least one, preferably at least two, more preferably at least three, even more preferably at least four, particularly preferred at least five and most preferred six X-ray powder diffraction peaks selected from the group consisting of 5.0, 7.7, 8.2, 10.0, 11.0, 15.4, 16.8, 17.7, 19.2, 20.5, 21.8, 26.5, 27.6 degrees two-theta+/−0.3 degrees two-theta, preferably +/−0.2 degrees two-theta, more preferably +/−0.1 degrees two-theta, most preferably +/−0.05 degrees two-theta.

85. The solid form of Tenofovir disoproxil and an organic acid according to claim 63, which is Tenofovir disoproxil salicylate TDSY ULT-1, wherein:
at least one, preferably at least two, more preferably at least three, even more preferably at least four, particularly preferred at least five and most preferred six X-ray powder diffraction peaks selected from the group consisting of 3.9, 5.1, 6.5, 9.7, 15.2, 16.3, 17.8, 19.0, 21.7, 22.4, 24.0, 27.3 degrees two-theta+/−0.3 degrees two-theta, preferably +/−0.2 degrees two-theta, more preferably +/−0.1 degrees two-theta, most preferably +/−0.05 degrees two-theta.

86. A pharmaceutical formulation comprising one or more forms of Tenofovir FD selected from the group consisting of Tenofovir disoproxil succinate TDSU ULT-1, Tenofovir disoproxil succinate TDSU ULT-2, Tenofovir disoproxil succinate TDSU ULT-3, Tenofovir disoproxil L-tartrate TDTA ULT-1, Tenofovir disoproxil L-tartrate TDTA ULT-2, Tenofovir disoproxil L-tartrate TDTA ULT-3, Tenofovir disoproxil L-tartrate TDTA ULT-4, Tenofovir disoproxil oxalate TDOX ULT-1, Tenofovir disoproxil oxalate TDOX ULT-2, Tenofovir disoproxil oxalate TDOX ULT-3, Tenofovir disoproxil oxalate TDOX ULT-4, Tenofovir disoproxil saccharate TDSA ULT-1, Tenofovir disoproxil saccharate TDSA ULT-2 Tenofovir disoproxil saccharate TDSA ULT-3, Tenofovir disoproxil citrate TDCI ULT-1, Tenofovir disoproxil salicylate TDSY ULT-1.

87. Use of one or more selected from the group consisting of Tenofovir disoproxil succinate TDSU ULT-1, Tenofovir disoproxil succinate TDSU ULT-2, Tenofovir disoproxil succinate TDSU ULT-3, Tenofovir disoproxil L-tartrate TDTA ULT-1, Tenofovir disoproxil L-tartrate TDTA ULT-2, Tenofovir disoproxil L-tartrate TDTA ULT-3, Tenofovir disoproxil L-tartrate TDTA ULT-4, Tenofovir disoproxil oxalate TDOX ULT-1, Tenofovir disoproxil oxalate TDOX ULT-2, Tenofovir disoproxil oxalate TDOX ULT-3, Tenofovir disoproxil oxalate TDOX ULT-4, Tenofovir disoproxil saccharate TDSA ULT-1, Tenofovir disoproxil saccharate TDSA ULT-2 Tenofovir disoproxil saccharate TDSA ULT-3, Tenofovir disoproxil citrate TDCI ULT-1, Tenofovir disoproxil salicylate TDSY ULT-1 as a medicament.

88. Use of one or more selected from the group consisting of Tenofovir disoproxil succinate TDSU ULT-1, Tenofovir disoproxil succinate TDSU ULT-2, Tenofovir disoproxil succinate TDSU ULT-3, Tenofovir disoproxil L-tartrate TDTA ULT-1, Tenofovir disoproxil L-tartrate TDTA ULT-2, Tenofovir disoproxil L-tartrate TDTA ULT-3, Tenofovir disoproxil L-tartrate TDTA ULT-4, Tenofovir disoproxil oxalate TDOX ULT-1, Tenofovir disoproxil oxalate TDOX ULT-2, Tenofovir disoproxil oxalate TDOX ULT-3, Tenofovir disoproxil oxalate TDOX ULT-4, Tenofovir disoproxil saccharate TDSA ULT-1, Tenofovir disoproxil saccharate TDSA ULT-2 Tenofovir disoproxil saccharate TDSA ULT-3, Tenofovir disoproxil citrate TDCI ULT-1, Tenofovir disoproxil salicylate TDSY ULT-1 in the treatment of HIV.

89. Use of one or more selected from the group consisting of Tenofovir disoproxil succinate TDSU ULT-1, Tenofovir disoproxil succinate TDSU ULT-2, Tenofovir disoproxil succinate TDSU ULT-3, Tenofovir disoproxil L-tartrate TDTA ULT-1, Tenofovir disoproxil L-tartrate TDTA ULT-2, Tenofovir disoproxil L-tartrate TDTA ULT-3, Tenofovir disoproxil L-tartrate TDTA ULT-4, Tenofovir disoproxil oxalate TDOX ULT-1, Tenofovir disoproxil oxalate TDOX ULT-2, Tenofovir disoproxil oxalate TDOX ULT-3, Tenofovir disoproxil oxalate TDOX ULT-4, Tenofovir disoproxil saccharate TDSA ULT-1, Tenofovir disoproxil saccharate TDSA ULT-2 Tenofovir disoproxil saccharate TDSA ULT-3, Tenofovir disoproxil citrate TDCI ULT-1, Tenofovir disoproxil salicylate TDSY ULT-1 in combination with another pharmaceutical ingredient, preferably an anti HIV agent, preferably Efavirenz and/or Emtricitabine.
